SEQUENCE LISTING (19 sequences)

SEQ ID NO: 1 - DNA, 588 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggatgagcttcacttccttgaaggagctcagcccgtttctcgttctggatgcattaaagagataaatgagtttgttggtacaaaacatgacccactaataccaacaaagagaaggaggcacagatcttgccgtctttttcggtggatcggatcaaaattgtgtatctgcatttcatgtctttgctactgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatctag

SEQ ID NO: 2 - DNA, 888 bp, Triticum aestivum
atgggggagggcgcggtggtggtgctggaggcgcccaagcccaggtcgccgccgaggtacccggacatgtgcggccgccggcgcctgcagctggaggtgcagatccttgaccgcgagctcacgttcctcaaggacgagctacatttacttgaaggggctcaaccagtctcacgttctggttgcttgaaagaggtaaacgagtttgtcggtacaaaacaagacccgctaataccaattaacaaaaggaagcaccggtcctgccgtctttattggtggatcagatcgaaactgtgcatatgtgcttcatggctgtgctgctcctgccaatgcctaccaacctgcaaaagaccaaggtgcttcgactgttcatgctgcgagccaaactgctcgtgctgcagcctgaactgctgcagctgcttcagtatcccttcgtgctgcaaaccaagctgtggctgctttgagtgctgcagctgcagcaaaccacagtgctgcagcagcggctgtaacccttgcggcgagtgcaagccggagtgcggctcgtgttccggcggcggctgctgcggcgacagcaggccaagctgcagctgctgcggcgagcagtgctgctcctgccctcgatgcacaggctgcttcagctgcttcaaggtccccaaatgctcgtgcgcgcagtgcttcaactgccagtcgtcgtgctgcaaggggcagccgtcgtgcttcaggtgccagtcgtcgtgctgcgacaagggaggctgctgcagcggcgggtcgtgcctgagctgcccgaagccgtcgtgcccggagtgctcctgcgggtgcgtgtggtcgtgcaaaaactgtacagacggatgccgatgcgcccggtgctgtgctggcgggtgcctgtgttaa

SEQ ID NO: 3 - DNA, 852 bp, Triticum urartu
atgggggagggcgcggtggtggtgctggaggcgcccaagcccaggtcgccgccgaggtacccggacatgtgcggccgccggcgcctgcagctggaggtgcagatccttgaccgcgagctcacgttcctcaaggacgagctacatttacttgaaggggctcaaccagtctcacgttctgcttgcttgaaagaggtaaatgagtttgttggtacaaaacaagatccactgataccaattaacaaaaggaagcatcggtcctgccgtctttattggtggatcagatcaaaactgtgcgtatgtgcttcatggctgtgctgctcctgccaatgcctaccaacctgcaaaagaccaagctgcttggactgttcatgctgcgagccaaactgctcgtgctgcagcccgaactgccgcagctgcttcaagatcccttcgtgctgcagcaaaccacagtgctgcagcggcggctgtaacccttgcggcgagtgcaagccggagtgtggctcttgttccgccggtggctgctgcggcgactgcaagccaagctgcagctgctgcggcgagcagtgccagtgctgctcctgccctcgatgcacggggggctgcttcaagctcccgaaatgctcgtgcgcgcagtgcttcaactgccagtcgtcgtgctgcaaggggcagccgtcgtgcttcaggtgccagtcgtcgtgctgcgacaagggcggctgctgcagcagcgggtcgtgcctgagctgcccgaagccgtcgtgcccggagtgctcctgcgggtgcgtgtggtcgtgcaaaaactgtacagacggatgccgatgcgcccggtgctgtgctggcgggtgcctgtgttaa

SEQ ID NO: 4 - DNA, 890 bp, Hordeum vulgare
atatgggggagggcgcggtggtggtgctggagccgcccaagcccaggtcgccgccgaggtacccggacatgtgcggccgccggcgcctgcagctggaggtgcagatccttgaccgcgagctcacgttcctcaaggacgagctacatttacttgaaggggctcaaccagtctcacgttctgcttgcttgaaagaggtaaatgagtttgttggtacaaaacaagatccactgataccaattaacaaaaggaagcatcggtcctgccgtctttattggtggatcagatcaaaactgtgcgtatgtgcttcatggctgtgctgctcctgccaatgcctaccaacctgcaaaagaccaagctgcttggactgttcatgctgcgagccaaactgctcgtgctgcagcccgaactgctgcagctgcttcaagatcccttcgtgctgcaaaccgagctgcggctgcttcgggtgctgcagctgcagcaaaccacagtgctgcagcggcggctgtaacccttgcggcgagtgcaagccggagtgtggctcttgttccgccggtggctgctgcggcgactgcaagccaagctgcagctgctgcggcgagcagtgccagtgctgctcctgccctcgatgcacggggggctgcttcaagctcccgaaatgctcgtgcgcgcagtgcttcaactgccagtcgtcgtgctgcaaggggcagccgtcgtgcttcaggtgccagtcgtcgtgctgcgacaagggcggctgctgcagcagcgggtcgtgcctgagctgcccgaagccgtcgtgcccggagtgctcctgcgggtgcgtgtggtcgtgcaaaaactgtacagacggatgccgatgcgcccggtgctgtgctagcgggtgcctgtgttga

SEQ ID NO: 5 - DNA, 1353 bp, Zea mays
atggcggcaccgggaagcccgacgccgcgccggctcccacgcccgcgccaacgccgctgccgccagaaaagcctctcccgggagagtgctgcggcagcgggtgcgttcgctgcgtcggggacatatatttcgacgagctcgacgcgtacgacaaggccctcgccgcgcgcgcggcctcctcaggctccggcggcaaggacgactctgctgataccaagcccaaagaaggtgccaaatcctgaagtgcgcctctcatgtgacgagctacaattacttgaaggagttccaccagtttctagaagttgtaaagaggtaaatgactttgttggtactaaacaagacccactaataccgataacgaaraagacacacagatcatgccgtcttttttggtggatcagatcaaaactgtgcatatgcgtgccatggttctgctgttcctgccattgcrtgcccaastgcaaaaggccatgcttcttggattgctcctgctgctcatgcccagatctgtcatgctgctatcccagctgcaaatcatgcaacaaaccatgctttgggcccaacagctgctcatgttgcgacatatcatgccgcaagccagattgcccatcctgtacctcgagctgcagctcgtgctgcaatcggccatgtggcaacccagactgcagctcgtgctgcacctgcaatccatcatgctgtaaaccaagctgcaactcgtgctgcagaccaaactgtagctcgtgctgcaatctgtcatgctgcaaacccagctgcagctcatgcttctcatgctgtaaagcgaactccagctcatgctgtaaacccaaatgcagctgcttcaaggccctctcgtgctgcaaattacagtgcagcccaaactgctgcacttgcagtctcccgagctgctcaggctgcaacccctgcggcagttgcaagcaatgctgctcatgccccagcgactgctttgattgcaagccaaactgcggctgtttcggcgcgcaatgctgcagctgcgtgggatgctgctcgtgctcctgccctcggtgctcaagctgcttcggttgcttcaagtacttcaagtgctccaacctgttcgggtgctgctcctgcaagcagtgcttcaagtgccagtcctcgtgctgcaagggggcgccgtcctgctgcaaatgccagtcgtcgtgctgtgagggtgaagacggcagcagcagctgctggaggtcatgctgcagcgttccgaagccggattgccctgggtgttcgtgcgggtgcgtttggtcctgcaggaagtgtacagaagggtgccgatgttctgggtgccgcaaaccatgctgtgccaccggatgcttgtgctga

SEQ ID NO: 6 - DNA, 1227 bp, Zea mays
atgggggaggaggtggcggtggtgctggagccaccacggcccaagtcgccgccgaggtacccggacctgtgcggccgccggcggtcgcagctggagctgcagatgctcaaccgcgagatcgaattcctcaaggacgagctacaattacttgaaggagttccaccagtttctagaagttgtaaagaggtaattgactttgttggtactaaacaagacccactaataccgataacgaagaagacacacagatcatgccgtcttttttggtggatcagatcaaaactgtgcatatgcgtgccatggttctgctgttcctgccattgcatgcccaactgcaaaaggccatgcttcttggattgctcctgctgctcatgcccagatctgtcatgctgctatcccagctgcaaatcatgcaacaaaccatgctttgggcccaacagctgctcatgttgcgacatatcatgccgcaagccagattgcccatcctgtacctcgagctgcagctcgtgctgcaatcggccatgtggcaacccagactgcagctcgtgctgcacctgcaatccatcatgctgtaaaccaagctgcaactcgtgctgcagaccaaactgtagctcgtgctgcaatctgtcatgctgcaaacccagctgcagctcatgcttctcatgctgtaaagcgaactccagctcatgctgtaaacccaaatgcagctgcttcaaggccctctcgtgctgcaaattacagtgcagcccaaactgctgcacttgcagtctcccgagctgctcaggctgcaacccctgcggcagttgcaagcaatgctgctcatgccccagcgactgctttgattgcaagccaaactgcggctgtttcggcgcgcaatgctgcagctgcgtgcgatgctgctcgtgctcctgccctcggtgctcaagctgcttcggttgcttcaagtacttcaagtgctccaacctgttcgggtgctgctcctgcaagcagtgcttcaagtgccagtcctcgtgctgcaagggggcgccgtcctgctgcaaatgccagtcgtcgtgctgtgagggtgaagacggcagcagcagctgctggaggtcatgctgcagcgttccgaagccggattgccctgggtgttcgtgcgggtgcgtttggtcctgcaggaagtgtacagaagggtgccgatgttctgggtgccgcaaaccatgctgtgccaccggatgcttgtgctga

SEQ ID NO: 7 - DNA, 1203 bp, Sorghum bicolor
atgggggaggaggtagcggcggcggcggcggtggtgctggagccgccacggcccaagtcgccgccgaggtacccggacctgtgcggccgccggcggctgcagctggagctccagatcctcaaccgcgagatcgacttcctcaaggacgagctacaatcactagaaggagttccaccagtttctagaagttgtaaagaggtaaatgagtttgttggtacaaaacaagacccactactaccgataaagaagaagacgcacagatcttgccgccttttttggtggatcagatcaaaattgtgcatatgcgtgtcatggttctgctgctcctgccattgcttgcccaactgcaaaagaccatgctgcttggattgctcctgctgctcatgcccagatctgtcatgctgcaagcccagctgcaaatcatgcaacaagccctgctcttggcccaacagctgttcatgttgtgacacaccatgctgcaagccagattgcccatcctgtagttcgagctgcagctcgtgctgcaatctgtcgtgttgcaacccgaactgtagctcgtgctgcacctgcaatccatcatgctgtaaaccaaactgcaactcgtgctgcagaccaaactgtagctcgtgctgcaatccgtcatgctgcaaacctaactgcggctcatggtggaaaccgagctgcagctgcttcaaggccccctcgtgctgcaaactccagtgcagccccaactgctgcacttgcagcctcccgcgctgctccggctgcaacccctgcggcagttgcaagcagtgttgctcatgccccaccgactgctgcaactgcaagccaagctgcggctgtttcagcgcgcaatgttgcagctgtgcggcatgctgctcctcgtgcttaagctgcttcagttgcttcggttgcttcaagtccttcaagtgctccaacctgttcgggtgctgctcctgcaagcagtgcttcaagtgccagtcgtcgtgctgcaagggtgcgccatcctgctgcaagtgccagtcgtcgtgctgtgagggtgaagacggcagcagctgctgccggaggtcatgctgcagtgttccgaagccggcttgccctgggtgctcgtgcgggtgcgtttggtcctgcaagaagtgtacagatgggtgtcgatgttctgggtgccgcaatccgtgctgtgccactggatgcttgtgttga

SEQ ID NO: 8 - DNA, 1281 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggatgagcttcacttccttgaaggagctcagcccgtttctcgttctggatgcattaaagagataaatgagtttgttggtacaaaacatgacccactaataccaacaaagagaaggaggcacagatcttgccgtctttttcggtggatcggatcaaaattgtgtatctgcatttcatgtctttgctactgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacagctgcttcaagatcccttcatgcttcaagtcccagtgcaactgctctagccccaattgctgcacttgcacccttccaagctgtagctgcaagggctgtgcctgtccaagctgtggatgcaacggctgtggctgtccaagctgcggatgcaacggttgtggctgtccaagctgcggttgcaacggctgtggccttccaagctgcggttgcaacggctgcggctcgtgctcttgcgcccaatgcaaacccgattgtggctcgtgctctaccaattgctgtagctgcaagccaagctgcaacggctgctgcggcgagcagtgctgccgctgcgcggactgcttctcctgctcgtgccctcgttgctccagctgcttcaacatcttcaaatgctcctgcgctggctgctgctcgagcctgtgcaagtgcccctgcacgacgcagtgcttcagctgccagtcgtcatgctgcaagcggcagccttcgtgctgcaagtgccagtcgtcttgctgcgaggggcagccttcctgctgcgagggacactgctgcagcctcccgaaaccgtcgtgccctgaatgttcctgtgggtgtgtctggtcttgcaagaattgtacagagggttgtcgatgcccacggtgtcgtaacccatgctgtctcagtggttgcttatgttga

SEQ ID NO: 9 - protein, 195 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNCSCSCPSCSSCCDTSCCKPSCTCFNI

SEQ ID NO: 10 - protein, 295 aa, Triticum aestivum
MGEGAVVVLEAPKPRSPPRYPDMCGRRRLQLEVQILDRELTFLKDELHLLEGAQPVSRSGCLKEVNEFVGTKQDPLIPINKRKHRSCRLYWWIRSKLCICASWLCCSCQCLPTCKRPRCFDCSCCEPNCSCCSLNCCSCFSIPSCCKPSCGCFECCSCSKPQCCSSGCNPCGECKPECGSCSGGGCCGDSRPSCSCCGEQCCSCPRCTGCFSCFKVPKCSCAQCFNCQSSCCKGQPSCFRCQSSCCDKGGCCSGGSCLSCPKPSCPECSCGCVWSCKNCTDGCRCARCCAGGCLC

SEQ ID NO: 11 - protein, 283 aa, Triticum urartu
MGEGAVVVLEAPKPRSPPRYPDMCGRRRLQLEVQILDRELTFLKDELHLLEGAQPVSRSACLKEVNEFVGTKQDPLIPINKRKHRSCRLYWWIRSKLCVCASWLCCSCQCLPTCKRPSCLDCSCCEPNCSCCSPNCRSCFKIPSCCSKPQCCSGGCNPCGECKPECGSCSAGGCCGDCKPSCSCCGEQCQCCSCPRCTGGCFKLPKCSCAQCFNCQSSCCKGQPSCFRCQSSCCDKGGCCSSGSCLSCPKPSCPECSCGCVWSCKNCTDGCRCARCCAGGCLC

SEQ ID NO: 12 - protein, 295 aa, Hordeum vulgare
MGEGAVVVLEPPKPRSPPRYPDMCGRRRLQLEVQILDRELTFLKDELHLLEGAQPVSRSACLKEVNEFVGTKQDPLIPINKRKHRSCRLYWWIRSKLCVCASWLCCSCQCLPTCKRPSCLDCSCCEPNCSCCSPNCCSCFKIPSCCKPSCGCFGCCSCSKPQCCSGGCNPCGECKPECGSCSAGGCCGDCKPSCSCCGEQCQCCSCPRCTGGCFKLPKCSCAQCFNCQSSCCKGQPSCFRCQSSCCDKGGCCSSGSCLSCPKPSCPECSCGCVWSCKNCTDGCRCARCCASGCLC

SEQ ID NO: 13 - protein, 170 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNC

SEQ ID NO: 14 - protein, 450 aa, Zea mays
MAAPGSPTPRRLPRPRQRRCRQKSLSRESAAAAGAFAASGTYISTSSTRTTRPSPRARPPQAPAARTTLLIPSPKKVPNPEVRLSCDELQLLEGVPPVSRSCKEVNDFVGTKQDPLIPITKKTHRSCRLFWWIRSKLCICVPWFCCSCHCMPNCKRPCFLDCSCCSCPDLSCCYPSCKSCNKPCFGPNSCSCCDISCRKPDCPSCTSSCSSCCNRPCGNPDCSSCCTCNPSCCKPSCNSCCRPNCSSCCNLSCCKPSCSSCFSCCKANSSSCCKPKCSCFKALSCCKLQCSPNCCTCSLPSCSGCNPCGSCKQCCSCPSDCFDCKPNCGCFGAQCCSCVGCCSCSCPRCSSCFGCFKYFKCSNLFGCCSCKQCFKCQSSCCKGAPSCCKCQSSCCEGEDGSSSCWRSCCSVPKPDCPGCSCGCVWSCRKCTEGCRCSGCRKPCCATGCLC

SEQ ID NO: 15 - protein, 408 aa, Zea mays
MGEEVAVVLEPPRPKSPPRYPDLCGRRRSQLELQMLNREIEFLKDELQLLEGVPPVSRSCKEVIDFVGTKQDPLIPITKKTHRSCRLFWWIRSKLCICVPWFCCSCHCMPNCKRPCFLDCSCCSCPDLSCCYPSCKSCNKPCFGPNSCSCCDISCRKPDCPSCTSSCSSCCNRPCGNPDCSSCCTCNPSCCKPSCNSCCRPNCSSCCNLSCCKPSCSSCFSCCKANSSSCCKPKCSCFKALSCCKLQCSPNCCTCSLPSCSGCNPCGSCKQCCSCPSDCFDCKPNCGCFGAQCCSCVRCCSCSCPRCSSCFGCFKYFKCSNLFGCCSCKQCFKCQSSCCKGAPSCCKCQSSCCEGEDGSSSCWRSCCSVPKPDCPGCSCGCVWSCRKCTEGCRCSGCRKPCCATGCLC

SEQ ID NO: 16 - protein, 400 aa, Sorghum bicolor
MGEEVAAAAAVVLEPPRPKSPPRYPDLCGRRRLQLELQILNREIDFLKDELQSLEGVPPVSRSCKEVNEFVGTKQDPLLPIKKKTHRSCRLFWWIRSKLCICVSWFCCSCHCLPNCKRPCCLDCSCCSCPDLSCCKPSCKSCNKPCSWPNSCSCCDTPCCKPDCPSCSSSCSSCCNLSCCNPNCSSCCTCNPSCCKPNCNSCCRPNCSSCCNPSCCKPNCGSWWKPSCSCFKAPSCCKLQCSPNCCTCSLPRCSGCNPCGSCKQCCSCPTDCCNCKPSCGCFSAQCCSCAACCSSCLSCFSCFGCFKSFKCSNLFGCCSCKQCFKCQSSCCKGAPSCCKCQSSCCEGEDGSSCCRRSCCSVPKPACPGCSCGCVWSCKKCTDGCRCSGCRNPCCATGCLC

SEQ ID NO: 17 - protein, 400 aa, Sorghum bicolor
MGEEVAAAAAVVLEPPRPKSPPRYPDLCGRRRLQLELQILNREIDFLKDELQSLEGVPPVSRSCKEVNEFVGTKQDPLLPIKKKTHRSCRLFWWIRSKLCICVSWFCCSCHCLPNCKRPCCLDCSCCSCPDLSCCKPSCKSCNKPCSWPNSCSCCDTPCCKPDCPSCSSSCSSCCNLSCCNPNCSSCCTCNPSCCKPNCNSCCRPNCSSCCNPSCCKPNCGSWWKPSCSCFKAPSCCKLQCSPNCCTCSLPRCSGCNPCGSCKQCCSCPTDCCNCKPSCGCFSAQCCSCAACCSSCSSCFSCFGCFKSFKCSNLFGCCSCKQCFKCQSSCCKGAPSCCKCQSSCCEGEDGSSCCRRSCCSVPKPACPGCSCGCVWSCKKCTDGCRCSGCRNPCCATGCLC

SEQ ID NO: 18 - protein, 401 aa, Sorghum bicolor
MGEEVAAAAAVVVLEPPRPKSPPRYPDLCGRRRLQLELQILNREIDFLKDELQSLEGVPPVSRSCKEVNEFVGTKQDPLLPIKKKTHRSCRLFWWIRSKLCICVSWFCCSCHCLPNCKRPCCLDCSCCSCPDLSCCKPSCKSCNKPCSWPNSCSCCDTPCCKPDCPSCSSSCSSCCNLSCCNPNCSSCCTCNPSCCKPNCNSCCRPNCSSCCNPSCCKPNCGSWWKPSCSCFKAPSCCKLQCSPNCCTCSLPRCSGCNPCGSCKQCCSCPTDCCNCKPSCGCFSAQCCSCAACCSSCSSCFSCFGCFKSFKCSNLFGCCSCKQCFKCQSSCCKGAPSCCKCQSSCCEGEDGSSCCRRSCCSVPKPACTGCSCGCVWSCKKCTEGCRCSGCRNPCCATGCLC

SEQ ID NO: 19 - protein, 426 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNCSCSCPSCSSCCDTSCCKPSCTCFNIFSCFKSLYSCFKIPSCFKSQCNCSSPNCCTCTLPSCSCKGCACPSCGCNGCGCPSCGCNGCGCPSCGCNGCGLPSCGCNGCGSCSCAQCKPDCGSCSTNCCSCKPSCNGCCGEQCCRCADCFSCSCPRCSSCFNIFKCSCAGCCSSLCKCPCTTQCFSCQSSCCKRQPSCCKCQSSCCEGQPSCCEGHCCSLPKPSCPECSCGCVWSCKNCTEGCRCPRCRNPCCLSGCLC

(19) United States
(12) Patent Application Publication
(10) Pub. No.: US 2017/0016013 A1
(43) Pub. Date: Jan. 19, 2017

(54) NOVEL USE OF A DENSE AND ERECT PANICLE 1 GENE IN IMPROVING NITROGEN UTILIZATION EFFICIENCY

(71) Applicants: Syngenta Participations AG, Basel (CH); Institute of Genetics and Developmental Biology, Chinese Academy of Sciences, Beijing (CN)

(72) Inventors: Xiangdong Fu, Beijing (CN); Kun Wu, Beijing (CN); Qian Qian, Beijing (CN); Chengwei Zhang, Beijing (CN); Xueying Liu, Beijing (CN); Shuansuo Wang, Beijing (CN)

(21) Appl. No.: 15/222,214

(22) Filed: Jul. 28, 2016

Related U.S. Application Data
(63) Continuation of application No. 13/982,229, filed on Oct. 1, 2013, now abandoned.

(30) Foreign Application Priority Data
Jan. 27, 2011 (CN) 201110029759.9

(51) Int. Cl. C12N 15/82; C07K 14/415

(52) U.S. Cl. CPC C12N 15/8243; C07K 14/415

(57) ABSTRACT

The present invention provides methods of increasing nitrogen utilization efficiency (NUE) in a transgenic plant comprising the introduction of a nucleic acid encoding a dep1 polypeptide into a plant to produce a transgenic plant that expresses the nucleic acid to produce the dep1 polypeptide, thereby resulting in an increased NUE as compared with a control plant. Also provided are methods of increasing NUE in a plant comprising reducing the amount and/or activity of a DEP1 polypeptide.

### RELATED APPLICATION INFORMATION

This application is a continuation application of, and claims priority to, U.S. application Ser. No. 13/982,229, filed Oct. 1, 2013, which is a 35 U.S.C. §371 national phase application of International Application Serial No. PCT/US2012/022930, filed Jan. 27, 2012, which claims priority to Chinese Application No. 201110029759.9, filed Jan. 27, 2011, the disclosure of each of which are incorporated by reference herein in its entirety.

### FIELD OF THE INVENTION

The present invention relates to methods for increasing the efficiency of nitrogen absorption, assimilation and/or utilization in plants, in particular, methods of increasing plant yield at a given level of nitrogen input.

### BACKGROUND OF THE INVENTION

Nitrogen is one of the nutrients needed in large quantities for the growth of plants. Nitrogen fertilizer is the chemical fertilizer needed in the largest quantity in agricultural production and plays an important function in the yield of crops and in improving the quality of agricultural products. Since the 1950s and 1960s, the global use of nitrogen fertilizer has rapidly increased by almost 10-fold. The result is that most of the high-yield varieties of major crops grown during the past several decades are highly dependent on nitrogen and other nutrients. However, generally speaking, only a small amount of nitrogen fertilizer that is applied is utilized by plants; most is released into the air or lost in water, creating an increasingly deleterious impact on the environment.

Rice is an important food crop, which provides food to approximately half of the world's population. The quantity of nitrogen fertilizer used for rice production comprises 37% of the total quantity of nitrogen fertilizer consumed worldwide. As noted above, most nitrogen is not utilized by the plant, but is instead released into the environment and lost in the form of N₂, NO₂, etc., which causes atmospheric pollution and eutrophication of rivers and lakes. Nonetheless, the use of nitrogen fertilizer is increasing year by year, and yet the production of rice is not increasing dramatically. On the contrary, intensification of fertilizer use is subject to diminishing returns, quite apart from its detrimental effects on the environment. The goal of raising crop productivity while conserving environmental quality represents a major challenge. Unfortunately, however, the genetic basis of nitrogen absorption and utilization by plants is still largely unknown.

Thus, there is a need in the art to develop new plant varieties that have increased yield at a given input of nitrogenous fertilizer.

### SUMMARY OF THE INVENTION

Enhancing nitrogen use efficiency (NUE) has become an urgent priority for achieving more sustainable agriculture. The present invention is based, in part, on the identification of the quantitative trait locus qNGR9, which is synonymous with DENSE ERECT PANICLE 1 (DEP1), a gene that is known to control plant architecture. The inventors have discovered that qngr9/dep1 improves NUE and, further, helps plants to adapt to low nitrogen conditions, thereby increasing yield with lower applications of nitrogenous fertilizer. Thus, dep1 is promising for the molecular breeding of new varieties that are both high yielding and nitrogen use efficient.

Accordingly, as one aspect, the invention provides a method of increasing NUE in a transgenic plant, the method comprising introducing an isolated nucleic acid encoding a dep1 polypeptide into a plant to produce a transgenic plant that expresses the isolated nucleic acid to produce the dep1 polypeptide, thereby resulting in an increased NUE in the transgenic plant as compared with a control plant.

In representative embodiments, the method further comprises growing the plant under low nitrogen conditions.

In embodiments of the invention, the method results in an increased yield of the transgenic plant under low nitrogen conditions as compared with a control plant. Optionally, the low nitrogen conditions comprise the application of a reduced level of nitrogen fertilizer to the transgenic plant and/or growing the transgenic plant in a low nitrogen medium.

In some embodiments, the plant comprises in its genome the isolated nucleic acid encoding a dep1 polypeptide.

In additional embodiment, the method comprises:

(a) introducing the isolated nucleic acid into a plant cell to produce a transgenic plant cell; and

(b) regenerating a transgenic plant from the transgenic plant cell of (a), wherein the transgenic plant comprises in its genome the isolated nucleic acid encoding a dep1 polypeptide and has increased NUE.

In further embodiments, the method comprises:

(a) introducing the isolated nucleic acid into a plant cell to produce a transgenic plant cell;

(b) regenerating a transgenic plant from the transgenic plant cell of (a), wherein the transgenic plant comprises in its genome the isolated nucleic acid encoding a dep1 polypeptide; and

(c) selecting from a plurality of the transgenic plants of (b) a transgenic plant having increased NUE.

In particular embodiments the method further comprises obtaining a progeny plant derived from the transgenic plant, wherein the progeny plant comprises in its genome the isolated nucleic acid encoding a dep1 polypeptide and has increased NUE.

In embodiments of the invention, the plant is a monocotyledonous plant (e.g., rice, maize, wheat, barley, sorghum, oat, rye or sugar cane).

In embodiments of the invention, the plant is a dicotyledonous plant (e.g., soybean or Arabidopsis).

The foregoing and other aspects of the present invention will now be described in more detail with respect to other embodiments described herein.

### BRIEF DESCRIPTION OF THE DRAWINGS

FIGS. 1A-C show the effects of nitrogen fertilizer on plant growth and cell proliferation. (A) The morphological response of paddy-grown plants to the application of nitrogenous fertilizer. Low N: 60 kg/ha, high N: 300 kg/ha. (B) Typical appearance of a semi-dwarf indica rice variety carrying sd1. (C) Longitudinal sections of the uppermost internode of the plants shown in (B).

FIGS. 2A-G show the variation in nitrogen sensitivity between QZL2 and NJ6. (A) japonica variety QZL2 was insensitive to nitrogen fertilizer. (B) indica variety was highly sensitive to nitrogen fertilizer. (C) RIL-D04 behaved in a similar manner to QZL2. (D) RIL-D22 was sensitive to nitrogen fertilizer. (E) Genetic analysis based on a BC₂F₂ population derived from the cross QZL2×D22 identified qNGR9, a major quantitative locus responsible for nitrogen growth response mapping on chromosome 9. (F) The dominant qngr9 allele from QZL2 was associated with semi-dwarfism. (G) Although the NIL-ngr9 internode cells were longer than those in NIL-NGR9, the internode length in NIL-ngr9 was less than in NIL-NGR9 plants.

FIG. 3 demonstrates the technical process for the cloning of major QTL:qNGR9.

FIGS. 4A-B show the differential effect of qNGR9 and qngr9 on plant height. (A) The height of NIL-ngr9 and NIL-NGR9 plants (B) The contribution of each internode and panicle to overall plant height.

FIG. 5 shows the effect of the qngr9 allele on the expression of genes involved in determining cell cycle time. Expression data were determined using qRT-PCT on RNA extracted from young internodal tissues. A fragment of the rice actin3 gene was used as a reference.

FIGS. 6A-E show ngr9 helps plants adapt to low nitrogen conditions. (A) NIL-ngr9 seedlings were less sensitive to exogenously supplied GA than NIL-NGR9. (B) and (C) Neither culm length nor cell number was enhanced in NIL-ngr9 plants by the application of nitrogenous fertilizer. (D) In a hydroponic system, the roots of NIL-NGR9 seedlings grew longer under lower nitrogen conditions, whereas shoot biomass accumulation was suppressed. In contrast, the root to shoot biomass ratio of NIL-ngr9 plants was unaffected by the reduction in nitrogen availability. (E) NIL-ngr9 out-performed NIL-NGR9 for grain yield.

FIGS. 7A-C show the response to gibberellic acid treatment of NIL-ngr9 plants. (A) the performance of NIL-ngr9/eui and NIL-NGR9/eui paddy-grown plants. (B) the effect of the qngr9 allele on the gibberellin-mediated degradation of DELLA. 14-day-old seedlings were challenged with anti-SLR1 antibody. (C) Gibberellin-induced alpha-amylase production in the aleurone layer of the developing rice grain. 2 μM GA, was used for treatment.

FIGS. 8A-F show the improved lodging resistance of NIL-ngr9 plants. (A-D) Cross-sections of the fourth internode of a NIL-ngr9 plant. (B) and (D) represent magnified views of the boxed sections shown in (A) and (C). (E) Comparison of the bending moment at breaking of the fourth internode at 21 days after heading for NIL-ngr9 and NIL-NGR9 plants. (F) Lodging in paddy-grown NIL-NGR9 and NIL-ngr9 plants treated with 30, 60, 120, 180, 240 and 300 kg/ha nitrogen, respectively.

FIGS. 9A-I depict changes in gene expression in response to nitrogen availability. Comparison of expression patterns of genes involved in nitrogen uptake and assimilation in the roots. Seedlings were exposed for 14 days to a hydroponic solution containing either low (0.025 mM) or high (1 mM) concentrations of NH₄NO₃. Data given as mean±SE (n=6), and a fragment of the rice actin3 gene was used as a reference.

FIGS. 10A-I show changes in gene expression in response to nitrogen availability. Comparison of expression patterns of genes involved in nitrogen assimilation and remobilization in the leaf tissues. Data given as mean±SE (n=6), and a fragment of the rice actin3 gene was used as a reference.

FIGS. 11A-B show that the ngr9 gene can improve yield and efficiency of the utilization of nitrogen fertilizer in rice. (A) shows that when NIL-ngr9 rice is planted with the application of different levels of nitrogen, the variation in plant height is not significant. (B) shows the effect of different levels of nitrogen fertilizer on the growth of rice leaves. High nitrogen concentrations can improve leaf growth in NIL-SD1 and NIL-NGR9 rice, but leaf growth for NIL-sd1 rice is only partially sensitive to high levels of nitrogen. The impact of nitrogen fertilizer on leaf growth for NIL-ngr9 rice was insignificant.

FIG. 12 shows the differential effect of qNGR9 and qngr9 on harvest index. NIL-ngr9 plants raised as described in FIG. 6. Data given as mean±SE (n=288).

FIGS. 13A-C demonstrate nitrogen uptake and utilization efficiency of NIL-NGR9 and NIL-ngr9 plants. (A) The above-ground nitrogen content of mature paddy-grown plants. (B) The physiological nitrogen utilization efficiency (ratio of grain dry mass to total above-ground nitrogen at harvest). (C) The ratio of nitrogen present in the grain to total above-ground nitrogen. Data shown represent mean±SE (n=60).

FIGS. 14A-C illustrate map-based cloning of qNGR9. (A) A fine-scale map of the target region. The candidate region was narrowed to a ˜18.6 kb region between marker W13 and W18 on rice chromosome 9. The numbers below the line indicate the number of recombinants recovered between qNGR9 and the marker shown. (B) Allelic variation of the candidate gene at the Os09g0441700 locus. (C) allelic variation of the candidate gene at the Os09g0441900 locus.

FIG. 15 shows that transgenic plants expressing qngr9/dep1 were insensitive to nitrogen availability. The transgenic plants had a semi-dwarf stature and showed no tendency to elongate either the stem or the leaf when supplied with nitrogenous fertilizer.

FIG. 16 shows an alignment of the DEP1 (top; SEQ ID NO: 8) and dep1 (bottom; SEQ ID NO: 1) cDNAs.

FIG. 17 shows an alignment of the amino acid sequences of DEP1 (top; SEQ ID NO: 19) and dep1 (bottom; SEQ ID NO: 9).

FIG. 18 is a schematic showing the structural and functional features of the NGR9/DEP1 protein.

FIG. 19 shows the sub-cellular localization of a NGR9/DEP1-GFP fusion protein in tobacco cells following Agrobacterium-mediated transformation.

FIG. 20 demonstrates that ngr9/dep1 increased the net efficiency of photosynthesis. During the sprouting period of the near isogenic lines NIL-dep1 and NIL-DEP1, from 10 AM to 12 noon, leaf tissue was exposed to various light intensities (250, 500, 750, 1000, 1500, 1800, 2000, 2500, 2800 μmol photons m⁻² sec⁻¹), and for each respective light intensity, the net absorption (μmol m⁻² sec⁻¹) of CO₂ on the rice field was measured. The test results show that under different light intensities, NIL-dep1 clearly has higher photosynthetic efficiency than NIL-DEP1.

FIG. 21 shows an alignment of a number of DEP1/dep1 orthologs, from top to bottom: DEP1 (SEQ ID NO: 19), dep1 (SEQ ID NO: 9), HvDep1 (SEQ ID NO: 12), TaDep1 (SEQ ID NO: 10), SbDEP1 (SEQ ID NO: 16), ZmDep1-2 (SEQ ID NO: 18), and ZmDep1-1 (SEQ ID NO: 17).

FIG. 22 demonstrates the natural variation that exists in the amino acid sequence of the SbDEP1 protein in different varieties of sorghum. Numerals (1) to (4) indicate amino acid positions with natural variation. Top: SEQ ID NO: 17, middle: SEQ ID NO: 16, and bottom: SEQ ID NO: 18.

### DETAILED DESCRIPTION OF THE INVENTION

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

### I. DEFINITIONS

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim or the description of this invention is not intended to be interpreted to be equivalent to “comprising.”

The terms “Nitrogen Utilization Efficiency” (NUE) refers to the ability of the plant to absorb, assimilate and/or use nitrogen, e.g., from soil, water and/or nitrogen fertilizer. Various methods are known in the art for assessing NUE. As one example, NUE can be determined by the yield achieved at a given level of nitrogen input, which may be from any source, for example, nitrogen present in the soil or other medium in which the plant is growing, nitrogen in the form of nitrogen fertilizer, and the like. This indicator is sometimes referred to as “agricultural” NUE. As another measure of NUE, the ratio of the plant product (e.g., grain dry mass) to above-ground nitrogen in the plant can be determined (sometimes referred to as “physiological” NUE). In embodiments of the invention, agricultural and/or physiological NUE is increased in a plant, optionally under low nitrogen conditions, as compared with a suitable control plant (e.g., a plant that does not express a dep1 polypeptide and/or has not been transformed with a nucleic acid encoding a dep1 polypeptide). The nitrogen can be in any form, including organic and/or inorganic forms, including without limitation nitrate (such as ammonium nitrate, calcium nitrate and/or potassium nitrate), nitrite, ammonia, aqua ammonia, anhydrous ammonia, ammonium sulfate, diammonium phosphate, a low-pressure nitrogen solution, a pressureless nitrogen solution, urea and/or urea-ammonium nitrate (UAN). In representative embodiments, the nitrogen is in a form that is immediately available to the plant (e.g., ammonia and/or nitrate) and/or can be readily converted to a form that is available to the plant (e.g., urea).

A number of approaches and indices of NUE are used in the art. For example, see Rice: Nutrient Disorders & Nutrient Management, A. Dobermann and T. Fairhurst. Potash & Phosphate Institute (PPI), Potash & Phosphate Institute of Canada (PPIC), and International Rice Research Institute (IRRI), which provides details of the following five indices:

(1) Partial Factor Productivity (PFP) from Applied Nitrogen: PFP is a measure of how much yield is produced for each unit of N applied

PFP_{N}=kg grain/kg N applied

PFP_{N}=GY_{+N}/FN

Where GY_{+N} is the grain yield (kg/ha) and FN is the amount of fertilizer N applied (kg/ha).

(2) Agronomic Efficiency (AE) of Applied Nitrogen: AE is a measure of how much additional yield is produced for each unit of N applied.

AE_{N}=kg grain yield increase/kg N applied

AE_{N}=(GY_{+N}−GY_{0N})/FN

Where GY_{+N} is the grain yield in a treatment with N application; GY_{0N} is the grain yield in a treatment without N application; and FN is the amount of fertilizer N applied, all in kg/ha.

(3) Recovery Efficiency (RE) of Applied Nitrogen: RE is a measure of how much of the N that was applied was recovered and taken up by the crop.

RE_{N}=kg N taken up/kg N applied

RE_{N}=(UN_{+N}−UN_{0N})/FN

Where UN_{+N} is the total plant N uptake measured in aboveground biomass at physiological maturity (kg/ha) in plots that received applied N at the rate of FN (kg/ha); and UN_{0N} is the total N uptake without the addition of N.

(4) Physiological Efficiency (PE) of Applied Nitrogen: PE is a measure of how much additional yield is produced for each additional unit of N uptake.

PE_{N}=kg grain yield increase/kg fertilizer N taken up

PE_{N}=(GY_{+N}−GY_{0N})/(UN_{+N}−UN_{0N+})

Where GY_{+N} is the grain yield in a treatment with N application (kg/ha); GY_{0N} is the grain yield in a treatment without N application; and UN is the total N uptake (kg/ha) in the two treatments.

(5) Internal Efficiency (IE) of Nitrogen: IE addresses how much yield is produced per unit N taken up from both fertilizer and indigenous (e.g., soil) nutrient sources.

IE_{N}=kg grain/kg N taken up

IE_{N}=GY/UN

Where GY is the grain yield (kg/ha), and UN is the total N uptake (kg/ha).

In embodiments of the invention, the increase in NUE is based on any one or more of the indices described above.

The term “low nitrogen conditions” (and the like), as used herein, indicates that a relatively low level of external nitrogen is provided to the plant, e.g., from the growing medium (e.g., soil), water and/or nitrogen fertilizer. The nitrogen can be in any suitable form, including organic and/or inorganic forms. In representative embodiments, the nitrogen is in the form of nitrate (such as ammonium nitrate, calcium nitrate and/or potassium nitrate), nitrite, ammonia, aqua ammonia, anhydrous ammonia, ammonium sulfate, diammonium phosphate, a low-pressure nitrogen solution, a pressureless nitrogen solution, urea and/or urea-ammonium nitrate (UAN). In representative embodiments, the nitrogen is in a form that is immediately available to the plant (e.g., ammonia and/or nitrate) and/or can be readily converted to a form that is available to the plant (e.g., urea). Nitrogen levels can be assessed, for example, with respect to a reference value that can be based on any suitable parameter. To illustrate, “low nitrogen conditions” can be relative to a reference value that is based, for example, on standard agricultural practices (e.g., for that species, variety and/or geographic location) and/or the optimum nitrogen level for plant productivity, the latter optionally taking into consideration adverse effects of providing high levels of nitrogen to the plant such as increased cost and/or detrimental environmental effects. In representative embodiments, “low nitrogen conditions” refer to a level of nitrogen that is equal to or less than about 85%, 80%, 75%, 70%, 60%, 50%, 40%, 30% or less of the nitrogen level that is standard and/or optimum for that plant species, variety and/or geographical location, etc. Those skilled in the art will recognize that “low nitrogen conditions” may vary with the plant species, plant variety, nitrogen form, soil type, geographic location, timing, weather, cropping intensity and other parameters that are well within the level of skill in the art. In representative embodiments, “low nitrogen conditions” are the result of the application of a reduced level of nitrogen fertilizer and/or growing the plant in a low nitrogen medium (e.g., soil, water, etc.).

As used herein, the term “reduced level of nitrogen fertilizer” and the like refers to the application of a relatively low level of nitrogen fertilizer to the plant. For example, the amount of fertilizer that is applied to the plant during the growing season can be reduced. The nitrogen fertilizer can be provided in any form, including organic and/or inorganic forms. In representative embodiments, the nitrogen fertilizer is in the form of nitrate (such as ammonium nitrate, calcium nitrate and/or potassium nitrate), nitrite, ammonia, aqua ammonia, anhydrous ammonia, ammonium sulfate, diammonium phosphate, a low-pressure nitrogen solution, a pressureless nitrogen solution, urea and/or urea-ammonium nitrate (UAN). In representative embodiments, the nitrogen fertilizer is in a form that is immediately available to the plant (e.g., ammonia and/or nitrate) and/or can be readily converted to a form that is available to the plant (e.g., urea). The amount of nitrogen fertilizer that is applied can be assessed, for example, with respect to a reference value that can be based on any suitable parameter such as, for example, standard agricultural practice (e.g., for that species, variety and/or geographical location) and/or the optimum level of nitrogen fertilizer for plant productivity, the latter optionally taking into consideration adverse effects of providing high levels of nitrogen to the plant such as increased cost and/or detrimental environmental effects. In representative embodiments, a “reduced level of nitrogen fertilizer” refers to the application of less than or equal to about 300, 275, 250, 225, 200, 175, 150, 120, 100, 80, 60, 40 or 20 kilograms/hectare (kg/ha) or less nitrogen fertilizer (these values referring to the “net weight” of nitrogen added, not the weight of the fertilizer), which can be applied at one time or by two or more applications prior to and/or during the growing season. Those skilled in the art will recognize that a “reduced level of nitrogen fertilizer” may vary with the plant species, plant variety, nitrogen form, soil type, geographic location, timing, weather, cropping intensity and other parameters that are well within the level of skill in the art.

For example, those skilled in the art will appreciate that the amount of nitrogen fertilizer applied can vary with the soil type and, in particular, the organic matter content of the soil. In some embodiments, the amount of nitrogen fertilizer applied decreases as the soil organic matter level increases, with soil organic matter content generally defined, for example, as follows: low (less than about 3.1% organic matter), medium (from about 3.1 to 4.5% organic matter), high (from about 4.6% to 19% organic matter), and organic soils (greater than about 19% organic matter). Nitrogen is also known to generally be low or deficient in soils that are very low in organic matter content (including coarse-textured acid soils); soils with low indigenous nitrogen supplies (e.g., acid sulfate soils, saline soils, phosphorus-deficient soils, poorly drained wetland soils, where the amount of nitrogen mineralization and/or biological nitrogen fixation is low); and alkaline and calcareous soils with low soil organic matter and a high potential for NH₃ volatilization losses.

Further, as a general rule, hybrid varieties have a higher nitrogen demand than inbred varieties; and dry season increases the need for application of nitrogen fertilizer as compared with the wet season. In the case of rice, transplanted rice generally results in a greater need for nitrogen fertilizer than rice grown by direct seeding.

As used herein, a “low nitrogen medium” and similar terms refers to a medium used to grow the plant (e.g., soil) that is relatively low or deficient in nitrogen, e.g., as organic and/or inorganic nitrogen (e.g., as nitrate, nitrite and/or ammonium). In representative embodiments, the nitrogen is in a form that is immediately available to the plant (e.g., ammonia and/or nitrate) and/or can be readily converted to a form that is available to the plant (e.g., urea). The amount of nitrogen that is present in the medium can be assessed, for example, with respect to a reference value that can be based on any suitable parameter such as, for example, standard agricultural practice (e.g., for that species, variety and/or geographical location) and/or the optimum level of nitrogen for plant productivity, the latter optionally taking into consideration adverse effects of providing high levels of nitrogen to the plant such as increased cost and/or detrimental environmental effects. In representative embodiments, “low nitrogen medium” refers to a nitrogen level that is less than or equal to about 100, 80, 60, 50, 40, 30, 25, 20, 15, or 10 kg/ha or less available nitrogen in the growing medium. Those skilled in the art will recognize that a “low nitrogen medium” may vary with the plant species, plant variety, nitrogen form, soil type, geographic location, timing, weather, cropping intensity and other parameters that are well within the level of skill in the art.

An “increased yield” (and similar terms) as used herein refers to an enhanced or elevated production of a commercially and/or agriculturally important plant, plant biomass, plant part (e.g., roots, tubers, seed, leaves, fruit), plant material (e.g., an extract) and/or other product produced by the plant (e.g., a recombinant polypeptide) obtained by a method of the present invention (e.g., transformed with a nucleic acid encoding a dep1 polypeptide) as compared with a control plant or part thereof (e.g., a plant that has not been transformed with an isolated nucleic acid encoding a dep1 polypeptide and/or a plant that does not comprise a native dep1 allele).

The term “modulate” (and grammatical variations) refers to an increase or decrease.

As used herein, the terms “increase,” “increases,” “increased,” “increasing” and similar terms indicate an elevation of at least about 25%, 50%, 75%, 100%, 150%, 200%, 300%, 400%, 500% or more.

As used herein, the terms “reduce,” “reduces,” “reduced,” “reduction” and similar terms mean a decrease of at least about 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

As used herein, the term “heterologous” means foreign, exogenous, non-native and/or non-naturally occurring.

As used here, “homologous” means native. For example, a homologous nucleotide sequence or amino acid sequence is a nucleotide sequence or amino acid sequence naturally associated with a host cell into which it is introduced, a homologous promoter sequence is the promoter sequence that is naturally associated with a coding sequence, and the like.

As used herein a “chimeric nucleic acid,” “chimeric nucleotide sequence” or “chimeric polynucleotide” comprises a promoter operably linked to a nucleotide sequence of interest that is heterologous to the promoter (or vice versa). In particular embodiments, the “chimeric nucleic acid,” “chimeric nucleotide sequence” or “chimeric polynucleotide” comprises a nucleic acid encoding a dep1 polypeptide operably associated with a heterologous promoter. A “promoter” is a nucleotide sequence that controls or regulates the transcription of a nucleotide sequence (i.e., a coding sequence) that is operatively associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). The promoter region, including all the ancillary regulatory elements, typically contain between about 100 and 1000 nucleotides, but can be as long as 2 kb, 3 kb, 4 kb or longer in length. Promoters according to the present invention can function as constitutive and/or inducible regulatory elements and can further be tissue-specific or tissue-preferred promoters.

A “heterologous promoter” is a promoter that is heterologous (e.g., foreign) to the nucleotide sequence with which it is operatively associated. For example, according to the present invention, the dep1 coding sequence can be operatively associated with a heterologous promoter (e.g., a promoter that is not the native dep1 promoter sequence with which the dep1 coding sequence is associated in its naturally occurring state).

“Nucleotide sequence of interest” refers to any nucleotide sequence which, when introduced into a plant, confers upon the plant a desired characteristic, for example, increased NUE. The “nucleotide sequence of interest” can encode a polypeptide (e.g., a dep1 polypeptide and/or an antibody or antibody for reducing the amount and/or activity of a DEP1 polypeptide) and/or an inhibitory polynucleotide (e.g., a functional RNA for reducing DEP1 expression and/or the amount and/or activity of a DEP1 polypeptide).

A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers and the like.

By “operably linked” or “operably associated” as used herein, it is meant that the indicated elements are functionally related to each other, and are also generally physically related. For example, a promoter is operatively linked or operably associated to a coding sequence (e.g., nucleotide sequence of interest) if it controls the transcription of the sequence. Thus, the term “operatively linked” or “operably associated” as used herein, refers to nucleotide sequences on a single nucleic acid molecule that are functionally associated. Those skilled in the art will appreciate that the control sequences (e.g., promoter) need not be contiguous with the coding sequence, as long as they functions to direct the expression thereof. Thus, for example, intervening untranslated, yet transcribed, sequences can be present between a promoter and a coding sequence, and the promoter sequence can still be considered “operably linked” to the coding sequence.

By the term “express,” “expressing” or “expression” (or other grammatical variants) of a nucleic acid coding sequence, it is meant that the sequence is transcribed. In particular embodiments, the terms “express,” “expressing” or “expression” (or other grammatical variants) can refer to both transcription and translation to produce an encoded polypeptide.

“Wild-type” nucleotide sequence or amino acid sequence refers to a naturally occurring (“native”) or endogenous nucleotide sequence (including a cDNA corresponding thereto) or amino acid sequence.

The terms “nucleic acid,” “polynucleotide” and “nucleotide sequence” are used interchangeably herein unless the context indicates otherwise. These terms encompass both RNA and DNA, including cDNA, genomic DNA, partially or completely synthetic (e.g., chemically synthesized) RNA and DNA, and chimeras of RNA and DNA. The nucleic acid, polynucleotide or nucleotide sequence may be double-stranded or single-stranded, and further may be synthesized using nucleotide analogs or derivatives (e.g., inosine or phosphorothioate nucleotides). Such nucleotides can be used, for example, to prepare nucleic acids, polynucleotides and nucleotide sequences that have altered base-pairing abilities or increased resistance to nucleases. The present invention further provides a nucleic acid, polynucleotide or nucleotide sequence that is the complement (which can be either a full complement or a partial complement) of a nucleic acid, polynucleotide or nucleotide sequence of the invention. Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage.

The nucleic acids and polynucleotides of the invention are optionally isolated. An “isolated” nucleic acid molecule or polynucleotide is a nucleic acid molecule or polynucleotide that, by the hand of man, exists apart from its native environment and is therefore not a product of nature. An isolated nucleic acid molecule or isolated polynucleotide may exist in a purified form or may exist in a non-native environment such as, for example, a recombinant host cell. Thus, for example, the term “isolated” means that it is separated from the chromosome and/or cell in which it naturally occurs. A nucleic acid or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and polynucleotides of the invention can be considered to be “isolated.”

Further, an “isolated” nucleic acid or polynucleotide can be a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The “isolated” nucleic acid or polynucleotide can exist in a cell (e.g., a plant cell), optionally stably incorporated into the genome. According to this embodiment, the “isolated” nucleic acid or polynucleotide can be foreign to the cell/organism into which it is introduced, or it can be native to an the cell/organism, but exist in a recombinant form (e.g., as a chimeric nucleic acid or polynucleotide) and/or can be an additional copy of an endogenous nucleic acid or polynucleotide. Thus, an “isolated nucleic acid molecule” or “isolated polynucleotide” can also include a nucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid or polynucleotide is at least about 10%, 20%, 30%, 40%, 50%, 60%, 70%, 80%, 90%, 95% or more pure.

As used herein, the term “recombinant” nucleic acid, polynucleotide or nucleotide sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in the cell, i.e., capable of nucleic acid replication under its own control. The term “vector” includes both viral and nonviral (e.g., plasmid) nucleic acid molecules for introducing a nucleic acid into a cell in vitro, ex vivo, and/or in vivo, and is optionally an expression vector. A large number of vectors known in the art may be used to manipulate, deliver and express polynucleotides. Vectors may be engineered to contain sequences encoding selectable markers that provide for the selection of cells that contain the vector and/or have integrated some or all of the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences of interest (e.g., transgenes), e.g., two, three, four, five or more heterologous nucleotide sequences of interest.

Viral vectors have been used in a wide variety of gene delivery applications in cells, as well as living animal subjects. Plant viral vectors that can be used include, but are not limited to, Agrobacterium tumefaciens, Agrobacterium rhizogenes and geminivirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (e.g., delivery to specific tissues, duration of expression, etc.).

The term “fragment,” as applied to a nucleic acid or polynucleotide, will be understood to mean a nucleotide sequence of reduced length relative to the reference or full-length nucleotide sequence and comprising, consisting essentially of and/or consisting of contiguous nucleotides from the reference or full-length nucleotide sequence. Such a fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 16, 17, 18, 19, 20, 25, 30, 35, 40, 45, 50, 75, 100, 125, 150, 175, 200, 225, 250, 275, 300, 325, 350, 375, 400, 405, 410, 425, 450, 455, 460, 475, 500, 505, 510, 515 or 520 nucleotides (optionally, contiguous nucleotides) or more from the reference or full-length nucleotide sequence, as long as the fragment is shorter than the reference or full-length nucleotide sequence. In representative embodiments, the fragment is a biologically active nucleotide sequence, as that term is described herein.

A “biologically active” nucleotide sequence is one that substantially retains at least one biological activity normally associated with the wild-type nucleotide sequence, for example, encoding a dep1 polypeptide that increases NUE in a plant and/or confers a semi-dwarf phenotype. In particular embodiments, the “biologically active” nucleotide sequence substantially retains all of the biological activities possessed by the unmodified sequence. By “substantially retains” biological activity, it is meant that the nucleotide sequence retains at least about 50%, 60%, 75%, 85%, 90%, 95%, 97%, 98%, 99%, or more, of the biological activity of the native nucleotide sequence (and can even have a higher level of activity than the native nucleotide sequence).

Two nucleotide sequences are said to be “substantially identical” to each other when they share at least about 60%, 70%, 75%, 80%, 85%, 90%, 95%, 97%, 98%, 99% or even 100% sequence identity.

Two amino acid sequences are said to be “substantially identical” or “substantially similar” to each other when they share at least about 60%, 70%, 75%, 80%, 85%, 90%, 95%, 97%, 98%, 99% or even 100% sequence identity or similarity, respectively.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25, 3389-3402 (1997).

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

Two nucleotide sequences can also be considered to be substantially identical when the two sequences hybridize to each other under stringent conditions. A nonlimiting example of “stringent” hybridization conditions include conditions represented by a wash stringency of 50% Formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

The polypeptides of the invention are optionally “isolated.” An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell. The recombinant polypeptides of the invention can be considered to be “isolated.”

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material. In representative embodiments, the isolated polypeptide is a recombinant polypeptide produced using recombinant nucleic acid techniques. In embodiments of the invention, the polypeptide is a fusion protein.

The term “fragment,” as applied to a polypeptide, will be understood to mean an amino acid of reduced length relative to a reference polypeptide or the full-length polypeptide and comprising, consisting essentially of, and/or consisting of a sequence of contiguous amino acids from the reference or full-length polypeptide. Such a fragment according to the invention may be, where appropriate, included as part of a fusion protein of which it is a constituent. In some embodiments, such fragments can comprise, consist essentially of, and/or consist of polypeptides having a length of at least about 30, 40, 50, 60, 70, 80, 90, 100, 110, 120, 130, 140, 150, 160, 165, 170, 175, 180 or 190 amino acids (optionally, contiguous amino acids) from the reference or full-length polypeptide, as long as the fragment is shorter than the reference or full-length polypeptide. In representative embodiments, the fragment is biologically active, as that term is defined herein.

A “biologically active” polypeptide is one that substantially retains at least one biological activity normally associated with the wild-type polypeptide, for example, increasing NUE in a plant and/or conferring a semi-dwarf phenotype. In particular embodiments, the “biologically active” polypeptide substantially retains all of the biological activities possessed by the unmodified (e.g., native) sequence. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). Methods of measuring NUE are known in the art, with non-limiting and exemplary methods described herein.

“Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol.

The term “transformation” as used herein refers to the introduction of a heterologous and/or isolated nucleic acid into a cell. Transformation of a cell may be stable or transient. Thus, a transgenic plant cell, plant tissue, plant part and/or plant of the invention can be stably transformed or transiently transformed.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” (and similar terms) in the context of a polynucleotide introduced into a cell, means that the introduced polynucleotide is stably integrated into the genome of the cell (e.g., into a chromosome or as a stable-extra-chromosomal element). As such, the integrated polynucleotide is capable of being inherited by progeny cells and plants.

“Genome” as used herein includes the nuclear and/or plastid genome, and therefore includes integration of a polynucleotide into, for example, the chloroplast genome. Stable transformation as used herein can also refer to a polynucleotide that is maintained extrachromosomally, for example, as a minichromosome.

As used herein, the terms “transformed” and “transgenic” refer to any plant, plant cell, plant tissue (including callus), or plant part that contains all or part of at least one recombinant or isolated nucleic acid, polynucleotide or nucleotide sequence. In representative embodiments, the recombinant or isolated nucleic acid, polynucleotide or nucleotide sequence is stably integrated into the genome of the plant (e.g., into a chromosome or as a stable extra-chromosomal element), so that it is passed on to subsequent generations of the cell or plant.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs plant cell tissue cultures, plant calli, plant clumps, and the like. As used herein, “shoot” refers to the above ground parts including the leaves and stems.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ.

Any plant (or groupings of plants, for example, into a genus or higher order classification) can be employed in practicing the present invention including angiosperms or gymnosperms, monocots or dicots.

Exemplary plants include, but are not limited to corn (Zea mays), canola (Brassica napus, Brassica rapa ssp.), alfalfa (Medicago saliva), rice (Oryza sativa, including without limitation Indica and/or Japonica varieties), rape (Brassica napus), rye (Secale cereale), sorghum (Sorghum bicolor, Sorghum vulgare), sunflower (Helianthus annus), wheat (Triticum aestivum), soybean (Glycine max), tobacco (Nicotiana tobacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Malus pumila), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris). duckweed (Lemna), oats (Avena sativa), barley (Hordium vulgare), vegetables, ornamentals, conifers, and turfgrasses (e.g., for ornamental, recreational or forage purposes), and biomass grasses (e.g., switchgrass and miscanthus).

Vegetables include Solanaceous species (e.g., tomatoes; Lycopersicon esculentum), lettuce (e.g., Lactuea sativa), carrots (Caucus carota), cauliflower (Brassica oleracea), celery (apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita such as Hubbard squash (C. Hubbard), Butternut squash (C. moschata), Zucchini (C. pepo), Crookneck squash (C. crookneck), C. argyrosperma, C. argyrosperma ssp sororia, C. digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (dianthus caryophyllus), poinsettia (Euphorbia pulcherima), and chrysanthemum.

Conifers, which may be employed in practicing the present invention, include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turfgrass include but are not limited to zoysiagrasses, bentgrasses, fescue grasses, bluegrasses, St. Augustine grasses, bermudagrasses, bufallograsses, ryegrasses, and orchardgrasses.

Also included are plants that serve primarily as laboratory models, e.g., Arabidopsis.

In representative embodiments, the plant does not comprise a native dep1 gene. In representative embodiments, the plant does comprise a native dep1 gene.

In other particular embodiments, the plant does not comprise a native DEP1 gene. In other exemplary embodiments, the plant does comprise a native DEP1 gene.

In further representative embodiments, the plant is not a leguminous plant.

### II. METHODS OF INTRODUCING A DEP1 POLYPEPTIDE INTO A PLANT TO INCREASE NUE

The invention provides methods of introducing a dep1 polypeptide into a plant material, e.g., a plant, plant part (including callus) or plant cell (e.g., to express the dep1 polypeptide in the plant material). In representative embodiments, the method comprises transforming the plant material with a nucleic acid, expression cassette, or vector of the invention encoding the dep1 polypeptide. The plant can be transiently or stably transformed.

As one aspect, the invention encompasses a method of increasing nitrogen utilization efficiency (NUE) in a transgenic plant, the method comprising introducing a nucleic acid (e.g., an isolated nucleic acid) encoding a dep1 polypeptide into a plant to produce a transgenic plant that expresses the isolated nucleic acid to produce the dep1 polypeptide (e.g., in an amount effective to increase NUE), thereby resulting in increased NUE in the plant. The plant can be transiently or stably transformed. The increase in NUE can be assessed with respect to any relevant control plant, e.g., a plant that does not express a dep1 polypeptide, a plant that has not been transformed with a nucleic acid encoding a dep1 polypeptide, a plant that is transformed with an irrelevant nucleic acid, and the like. The control plant is generally matched for species, variety, age, and the like and is subjected to the same growing conditions, e.g., temperature, soil, sunlight, pH, water, and the like. The selection of a suitable control plant is routine for those skilled in the art.

In representative embodiments, there is an increase in yield of the transgenic plant (including the yield of a plant product) as compared with a control plant at a given level of nitrogen (e.g., from the growing medium and/or in the form of nitrogen fertilizer). In embodiments of the invention, there is an increase in the ratio of a plant product (e.g., the grain dry mass) to above-ground nitrogen in the transgenic plant as compared with a control plant, for example, at a given level of nitrogen (e.g., from the growing medium and/or in the form of nitrogen fertilizer).

The transgenic plant can be grown under conditions of standard, or even high, nitrogen conditions. In other exemplary embodiments, the transgenic plant is grown under low nitrogen conditions. The low nitrogen conditions can arise from the application of a reduced level of nitrogen fertilizer to the transgenic plant and/or growing the plant in a low nitrogen medium (e.g., soil, water, and the like). According to this embodiment, there is optionally an increase in yield of the transgenic plant (including the yield of a plant product) as compared with a control plant grown under the low nitrogen conditions. In embodiments of the invention, there is an increase in the ratio of a plant product (e.g., the grain dry mass) to above-ground nitrogen in the transgenic plant as compared with a control plant under the low nitrogen conditions.

In particular embodiments, the method comprises: (a) introducing the nucleic acid (e.g., isolated nucleic acid) encoding a dep1 polypeptide into a plant cell (including a callus cell) to produce a transgenic plant cell; and (b) regenerating a transgenic plant from the transgenic plant cell of (a), optionally wherein the transgenic plant comprises in its genome the isolated nucleic acid encoding a dep1 polypeptide and, as a further option, has increased NUE as compared with a control plant (e.g., expresses the dep1 polypeptide in an amount effective to increase NUE in the plant).

In representative embodiments, the method further comprises selecting from a plurality of the transgenic plants of (b) a transgenic plant having increased NUE.

The invention also contemplates the production of progeny plants that comprise the nucleic acid encoding a dep1 polypeptide. In embodiments of the invention, the method further comprises obtaining a progeny plant derived from the transgenic plant (e.g., by sexual reproduction or vegetative propagation), optionally wherein the progeny plant comprises in its genome the isolated nucleic acid encoding a dep1 polypeptide and has increased NUE as compared with a control plant (e.g., expresses the dep1 polypeptide in an amount effective to increase NUE in the plant).

To illustrate, in one embodiment, the invention provides a method of producing a progeny plant, the method comprising (a) crossing the transgenic plant comprising the nucleic acids encoding a dep1 polypeptide with itself or another plant to produce seed comprising the nucleic acid encoding the dep1 polypeptide; and (b) growing a progeny plant from the seed to produce a transgenic plant, optionally wherein the progeny plant comprises in its genome the isolated nucleic acid encoding a dep1 polypeptide and has increased NUE as compared with a control plant (e.g., expresses the dep1 polypeptide in an amount effective to increase NUE in the plant). In additional embodiments, the method can further comprise (c) crossing the progeny plant with itself or another plant and (d) repeating steps (b) and (c) for an additional 0-7 (e.g., 0, 1, 2, 3, 4, 5, 6 or 7 and any range thereof) generations to produce a plant, optionally wherein the plant comprises in its genome the isolated nucleic acid encoding a dep1 polypeptide and has increased NUE as compared with a control plant (e.g., expresses the dep1 polypeptide in an amount effective to increase NUE in the plant).

The term “dep1 polypeptide” is intended broadly and encompasses naturally occurring dep1 polypeptides and equivalents (including fragments) thereof and increase NUE in a plant. The term “dep1” polypeptide also includes modifications (e.g., deletions and/or truncations) of a naturally occurring DEP1 polypeptide or an equivalent thereof that has a substantially similar or identical amino acid sequence to a naturally occurring DEP1 polypeptide and that increases NUE in a plant. Further, the dep1 gene has been identified in a number of plant species, and the dep1 polypeptide can be from any plant species of origin (e.g., rice [including indica and/or japonica varieties], wheat, barley, maize, sorghum, oats, rye, sugar cane and the like), and the term “dep1 polypeptide” also includes naturally occurring allelic variations, isoforms, splice variants and the like. The dep1 polypeptide can further be wholly or partially synthetic.

Unless indicated otherwise, dep1 polypeptides of the invention include dep1 fusion proteins comprising a dep1 polypeptide of the invention. For example, it may be useful to express the dep1 polypeptide as a fusion protein that can be detected by a commercially available antibody (e.g., a FLAG motif) or as a fusion protein that can otherwise be more easily detected or purified (e.g., by addition of a poly-His tail). Additionally, fusion proteins that enhance the stability of the protein can be produced, e.g., fusion proteins comprising maltose binding protein (MBP) or glutathione-S-transferase. As another alternative, the fusion protein can comprise a reporter molecule.

The term “DEP1” polypeptide includes naturally occurring DEP1 polypeptides and equivalents thereof that have substantially similar or identical amino acid sequences to a naturally occurring DEP1 polypeptide. In general, DEP1 polypeptides do not confer increased NUE in a plant. In addition, DEP1 polypeptides typically do not confer a semi-dwarf phenotype in the plant. A number of native DEP1 polypeptides have previously been identified and include, for example, the polypeptides of SEQ ID NOS: 14-19. Variants of the rice DEP1 have also previously been described (see, e.g., U.S. Patent Application Publication 2011/0197305 A1). It would be routine to identify other DEP1 polypeptides and the genes encoding the same using standard methods known to those skilled in the art (e.g., homology based cloning) using the present application as a guide and the general knowledge in the art.

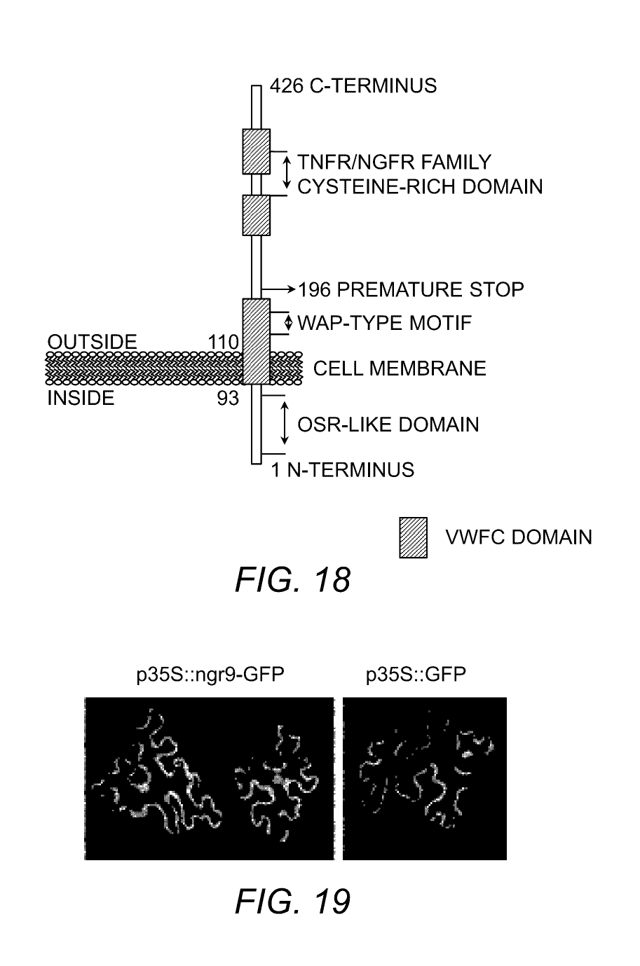

As shown in FIG. 18, the full-length rice DEP1 (e.g., SEQ ID NO: 19) is a transmembrane protein that comprises a number of structural/functional domains. In the N-terminal portion is an organ size regulation (OSR) like domain (e.g., within amino acids 1-80 of SEQ ID NO:19). In addition, a G-protein gamma like (GGL) domain has been identified in the N-terminal portion (e.g., from amino acids 28-82 of SEQ ID NO: 19). The rice DEP1 protein also has a single transmembrane domain (e.g., amino acids 93-110 of SEQ ID NO:19). In the extracellular portion of the protein is a Whey Acidic Protein (WAP)-type motif (e.g., amino acids 153-166 of SEQ ID NO: 19). The C-terminal portion of the protein comprises a TNFR/NGFR family cysteine-rich domain, which is entirely absent from the native rice dep1 proteins shown in SEQ ID NOS: 9 and 13. Further, there are three Von Willebrand Factor Type C (VWFC) domains from amino acids 99-153, 276-316 and 339-385. Only the first of these is conserved in the rice dep1 proteins of SEQ ID NOS: 9 and 13.

The importance of VWFC and TNFR/NGFR family cysteine-rich domains have previously been studied with respect to the rice GS3 locus (Fan et al., 2006 Theor Appl. Genet. 112: 1164-1171). The GS3 protein is a 232 amino acid protein having a putative OSR-like domain, a transmembrane region, TNFR/NGFR family cysteine-rich domain and a VWFC domain. A nonsense mutation resulting in a C-terminal truncation of 178 amino acids was identified in all large-grain rice varieties tested, but was absent from small grain varieties. This truncation resulted in a deletion of a portion of the OSR domain and complete loss of the transmembrane, TNFR/NGFR family cysteine-rich domain and the VWFC domain. These investigators suggest that GS3 is a negative regulator to prevent an increase in grain size. The authors note that a similar nonsense mutation/truncation and the loss of the VWFC domain appear to be involved in the determination of tomato fruit shape by the OVATE locus. The recessive pear shape (as opposed to the dominant round shape) is associated with a premature stop codon in the OVATE gene with a resulting deletion of the VWFC domain.

In rice dep1 (e.g., SEQ ID NO: 9), there is also a loss of a substantial portion of the C-terminal regions of the protein, including deletion of two of the VWFC domains and the TNFR/NGFR family cysteine-rich domain. Thus, this information can be used to modify a DEP1 polypeptide (including equivalents thereof) to construct dep1 polypeptides that enhance NUE in a plant and, optionally, produce a semi-dwarf phenotype.

In representative embodiments, modification of a DEP1 polypeptide to produce a dep1 polypeptide comprises deleting one or both of the two VWFC domains in the C-terminal portion of the protein (e.g., the VWFC domains at amino acids 276-316 and 339-385 of SEQ ID NO: 19). In embodiments of the invention, only a portion of the third VWFC domain (from the N-terminus) is deleted (e.g., at least about 10%, 20%, 30%, 40%, 50%, 60%, 70%, 80%, 90%, 95%, etc. is deleted). In additional embodiments, a portion of the second VWFC domain (from the N-terminus) is deleted (e.g., at least about 10%, 20%, 30%, 40%, 50%, 60%, 70%, 80%, 90%, 95%, etc. is deleted) and, optionally, the third VWFC domain (from the N-terminus) is deleted. In embodiments all three VWFC domains are deleted. In additional embodiments, the first VWFC domain (from the N-terminus) is partially deleted (e.g., at least about 10%, 20%, 30%, 40%, 50%, 60%, 70%, 80%, 90%, 95%, etc. is deleted) and, optionally, the second and/or third VWFC domains (from the N-terminus) are deleted.

In additional embodiments of the invention, modification of a DEP1 polypeptide to produce a dep1 polypeptide comprises deleting all of the TNFR/NGFR family cysteine-rich domain. In embodiments of the invention, modification of a DEP1 polypeptide to produce a dep1 polypeptide comprises deleting at least a portion of the TNFR/NGFR family cysteine-rich domain (e.g., at least about 10%, 20%, 30%, 40%, 50%, 60%, 70%, 80%, 90%, 95%, etc. is deleted).

In further embodiments of the invention, modification of a DEP1 polypeptide to produce a dep1 polypeptide comprises deleting all or a portion of the WAP-type motif (e.g., at least about 10%, 20%, 30%, 40%, 50%, 60%, 70%, 80%, 90%, 95%, etc. is deleted).

In additional exemplary embodiments, modification of a DEP1 polypeptide to produce a dep1 polypeptide comprises deleting at least about 10, 20, 30, 40, 50, 60, 70, 80, 100, 120, 140, 160, 180, 200, 220, 240, 260, 280, 300, 320, 340, 360 or 380 amino acids from the C-terminus of a DEP1 polypeptide (e.g., is a truncated protein), including any range therein, e.g., from about 10 to 20, 30, 40, 50, 60, 70, 80, 100, 120, 140, 160, 180, 200, 220, 240, 260, 280, 300, 320, 340, 360 or 380 amino acids are deleted from the C-terminus of a DEP1 polypeptide; at least about 20 to 30, 40, 50, 60, 70, 80, 100, 120, 140, 160, 180, 200, 220, 240, 260 or 280, 300, 320, 340, 360 or 380 amino acids are deleted from the C-terminus of a DEP1 polypeptide, at least about 30 to 40, 50, 60, 70, 80, 100, 120, 140, 160, 180, 200, 220, 240, 260, 280, 300, 320, 340, 360 or 380 amino acids are deleted from the C-terminus of a DEP1 polypeptide; at least about 40 to 50, 60, 70, 80, 100, 120, 140, 160, 180, 200, 220, 240, 260, 280, 300, 320, 340, 360 or 380 amino acids are deleted from the C-terminus of a DEP1 polypeptide; at least about 50 to about 60, 70, 80, 100, 120, 140, 160, 180, 200, 220, 240, 260, 280, 300, 320, 340, 360 or 380 amino acids are deleted from the C-terminus of a DEP1 polypeptide; at least about 60 to 70, 80, 100, 120, 140, 160, 180, 200, 220, 240, 260, 280, 300, 320, 340, 360 or 380 amino acids are deleted from the C-terminus of a DEP1 polypeptide; at least about 70 to 80, 100, 120, 140, 160, 180, 200, 220, 240, 260, 280, 300, 320, 340, 360 or 380 amino acids are deleted from the C-terminus of a DEP1 polypeptide; at least about 80 to 100, 120, 140, 160, 180, 200, 220, 240, 260, 280, 300, 320, 340, 360 or 380 amino acids are deleted from the C-terminus of a DEP1 polypeptide; at least about 100 to 120, 140, 160, 180, 200, 220, 240, 260, 280, 300, 320, 340, 360 or 380 amino acids are deleted from the C-terminus of a DEP1 polypeptide; at least about 120 140, 160, 180, 200, 220, 240, 260, 280, 300, 320, 340, 360 or 380 amino acids are deleted from the C-terminus of a DEP1 polypeptide; at least about 140 to 160, 180, 200, 220, 240, 260, 280, 300, 320, 340, 360 or 380 amino acids are deleted from the C-terminus of a DEP1 polypeptide; at least about 160 to 180, 200, 220, 240, 260, 280, 300, 320, 340, 360 or 380 amino acids are deleted from the C-terminus of a DEP1 polypeptide; at least about 180 to 200, 220, 240, 260, 280, 300, 320, 340, 360 or 380 amino acids are deleted from the C-terminus of a DEP1 polypeptide; at least about 200 to 220, 240, 260, 280, 300, 320, 340, 360 or 380 amino acids are deleted from the C-terminus of a DEP1 polypeptide; at least about 220 to 240, 260, 280, 300, 320, 340, 360 or 380 amino acids are deleted from the C-terminus of a DEP1 polypeptide, etc.

In further exemplary embodiments, modification of a DEP1 polypeptide to produce a dep1 polypeptide comprises deleting essentially all of the extracellular domain of the DEP1 polypeptide (e.g., at least about 10%, 20%, 30%, 40%, 50%, 60%, 70%, 80%, 90%, 95%, etc. of the extracellular domain is deleted), including any range therein. For example, according to particular embodiments, at least about 10, 20, 30, 40, 50, 60, 70, 80, 100, 120, 140, 160, 180, 200, 220, 240, 260, 280, 290, 300, 310 or 320 amino acids from the extracellular domain of a DEP1 polypeptide are deleted, including any range therein.

In representative embodiments, a dep1 polypeptide comprises one or more of the OSR, GGL, the first VWFC (from the N-terminus; e.g., amino acids 99-153 of SEQ ID NO: 19) and/or transmembrane domains from a DEP1 polypeptide. In representative embodiments, the dep1 polypeptide comprises the OSR and GGL domains, but the first VWFC is completely or partially deleted (e.g., at least about 10%, 20%, 30%, 40%, 50%, 60%, 70%, 80%, 90%, 95%, etc. is deleted). In additional embodiments, the dep1 polypeptide comprises all of the WAP-type motif or a portion thereof (e.g., at least about 10%, 20%, 30%, 40%, 50%, 60%, 70%, 80%, 90%, 95%, etc.). In embodiments of the invention, the dep1 polypeptide comprises only a portion of the OSR domain (e.g., less than about 10%, 20%, 30%, 40%, 50%, 60%, 70%, 80%, 90%, 95%, etc.).

In embodiments of the invention, the dep1 polypeptide comprises about the 10, 20, 30, 40, 50, 60, 70, 80, 100, 110, 120, 130, 140, 150, 160, 170, 180, 190, 200, 220, 240, 260, 280, 300, 320, 340, 360, 380, 400 or 420 N-terminal amino acids from a DEP1 polypeptide, including any range therein.

Those skilled in the art will appreciate that other modifications can be made (e.g., amino acid substitutions) to the one or more functional domains in a DEP1 polypeptide to reduce the activity thereof to produce a dep1 polypeptide. For example, one or more cysteines in the first, second and/or third VWFC domains can be replaced with another amino acid (e.g., a non-conservative amino acid). In other embodiments, one or more cysteines in the WAP domain and/or TNFR/NGFR family cysteine-rich domain can be substituted with another amino acid (e.g., a non-conservative amino acid).

A number of native dep1 polypeptides have been identified in the art from a variety of species (e.g., SEQ ID NOS: 9-13). It is well within the skill of those in the art to identify other dep1 polypeptides and the genes encoding the same using the present application as a guide and the knowledge in the art.

In particular embodiments, the dep1 polypeptide comprises, consists essentially of, or consists of the amino acid sequence of any of SEQ ID NOS: 9-13 or equivalents thereof (including fragments and equivalents thereof).

Equivalents of the dep1 polypeptides of the invention encompass those that have substantial amino acid sequence identity or similarity, for example, at least about 60%, 70%, 75%, 80%, 85%, 90%, 95%, 97%, 98%, 99% or more amino acid sequence identity or similarity with the amino acid sequence of a naturally occurring dep1 polypeptide (e.g., SEQ ID NOS: 9-13) or a fragment thereof, optionally a biologically active fragment.

In representative embodiments, the dep1 polypeptide comprises one or more of the OSR, GGL, the first VWFC (from the N-terminus; e.g., amino acids 99-153 of SEQ ID NO: 9) and/or transmembrane domains from a dep1 polypeptide and, optionally, any sequence variability occurs outside of these domains. In representative embodiments, the dep1 polypeptide comprises the OSR and GGL domains, but the first VWFC is completely or partially deleted (e.g., at least about 10%, 20%, 30%, 40%, 50%, 60%, 70%, 80%, 90%, 95%, etc. is deleted). In additional embodiments, the dep1 polypeptide comprises all of the WAP-type motif or a portion thereof (e.g., at least about 10%, 20%, 30%, 40%, 50%, 60%, 70%, 80%, 90%, 95%, etc.). In embodiments of the invention, the dep1 polypeptide does not comprise the WAP-type domain or at least a portion thereof is deleted (e.g., at least about 10%, 20%, 30%, 40%, 50%, 60%, 70%, 80%, 90%, 95%, etc. is deleted) from the dep1 polypeptide. In embodiments of the invention, the dep1 polypeptide comprises no extracellular domain or substantially no extracellular domain, e.g., the extracellular domain comprises about 1, 2, 3, 4, 5, 6, 7, 8, 9, 10, 12, 15, 20, 30, 40, 50 or 60 amino acids or less. In embodiments of the invention, the dep1 polypeptide comprises only a portion of the OSR domain (e.g., less than about 10%, 20%, 30%, 40%, 50%, 60%, 70%, 80%, 90%, 95%, etc.). In additional embodiments, the dep1 polypeptide comprises a C-terminal truncation of at least about 1, 2, 3, 4, 5, 10, 15, 20, 25, 30, 35, 40, 50, 60, 70, 80, 90, 100 or 125 amino acids as compared with a native dep1 polypeptide.

In further illustrative embodiments, the dep1 polypeptide comprises at least about 40, 50, 60, 70, 80, 90, 100, 110, 120, 130, 140, 150, 160 or 165 amino acids of a native dep1 polypeptide.

It will further be understood that naturally occurring dep1 polypeptides will typically tolerate substitutions in the amino acid sequence and substantially retain biological activity. To routinely identify biologically active dep1 polypeptides of the invention other than naturally occurring dep1 polypeptides (e.g., SEQ ID NOS: 9-13), amino acid substitutions may be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding the dep1 polypeptide.

In making amino acid substitutions, the hydropathic index of amino acids can be considered. The importance of the hydropathic amino acid index in conferring interactive biologic function on a protein is generally understood in the art (see, Kyte and Doolittle, (1982) J. Mol. Biol. 157:105). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

The dep1 polypeptides of the present invention also encompass dep1 polypeptide fragments that increase NUE in a plant, and equivalents thereof (optionally, biologically active equivalents). The length of the dep1 fragment is not critical. Illustrative dep1 polypeptide fragments comprise at least about 40, 50, 75, 100, 125, 150, 160, 161, 162, 163, 164, 165, 166, 167, 168, 169, 170, 175, 180, 185, 186, 187, 188, 189, 190, 191, 192, 193 or 194 amino acids (optionally, contiguous amino acids) of a dep1 polypeptide.

In representative embodiments, the dep1 polypeptide comprises, consists essentially of, or consists of an amino acid sequence selected from the group consisting of: (a) the amino acid sequence of any of SEQ ID NOS: 9-13; (b) an amino acid sequence having at least about 60%, 70%, 75%, 80%, 85%, 90%, 95%, 97%, 98%, 99% or more amino acid sequence identity or similarity with the amino acid sequence of any of SEQ ID NOS: 9-13, optionally wherein the dep1 polypeptide is biologically active; and (c) a fragment comprising at least about 40, 50, 75, 100, 125, 150, 160, 161, 162, 163, 164, 165, 166, 167, 168, 169, 170, 175, 180, 185, 186, 187, 188, 189, 190, 191, 192, 193 or 194 amino acids (optionally, contiguous amino acids) amino acids of the amino acid sequence of (a) or (b) above, wherein the fragment increases NUE in a plant.

Nucleic acids encoding dep1 polypeptides of the invention can be from any species of origin (e.g., plant species) or can be partially or completely synthetic. In representative embodiments, the nucleic acid encoding the dep1 polypeptide is an isolated nucleic acid.

Nucleic acids encoding dep1 polypeptides have been identified from a number of species including rice, wheat (e.g., T. aestivum and T. urartu) and barley (see, e.g., SEQ ID NOS: 1-4). Orthologs from other organisms, in particular other plants, can be routinely identified using methods known in the art. For example, PCR and other amplification techniques and hybridization techniques can be used to identify such orthologs based on their sequence similarity to the sequences set forth herein.

In representative embodiments, the nucleotide sequence encoding the dep1 polypeptide is a naturally occurring nucleotide sequence (e.g., SEQ ID NOS: 1-4) or encodes a naturally occurring dep1 polypeptide (e.g., SEQ ID NOS: 9-13), or is a nucleotide sequence that has substantial nucleotide sequence identity thereto and which encodes a biologically active dep1 polypeptide.

The invention also provides polynucleotides encoding the dep1 polypeptides of the invention, wherein the polynucleotide hybridizes to the complete complement of a naturally occurring nucleotide sequence encoding a dep1 polypeptide (e.g., SEQ ID NOS: 1-4) or a nucleotide sequence that encodes a naturally occurring dep1 polypeptide (e.g., SEQ ID NOS: 9-13) under stringent hybridization conditions as known by those skilled in the art and encode a biologically active dep1 polypeptide.

Further, it will be appreciated by those skilled in the art that there can be variability in the polynucleotides that encode the dep1 polypeptides due to the degeneracy of the genetic code. The degeneracy of the genetic code, which allows different nucleotide sequences to code for the same protein, is well known in the art. Moreover, plant or species-preferred codons can be used in the polynucleotides encoding the dep1 polypeptides, as is also well-known in the art.

In exemplary, but non-limiting, embodiments, the nucleic acid (e.g., recombinant or isolated nucleic acid) encoding a dep1 polypeptide comprises, consists essentially of, or consists of a nucleotide sequence selected from the group consisting of: (a) a nucleotide sequence comprising the nucleotide sequence of any of SEQ ID NOS: 1-4; (b) a nucleotide sequence comprising at least about 50, 75, 100, 125, 150, 175, 200, 225, 250, 275, 300, 325, 350, 375, 400, 425, 450, 475 or 500 or more nucleotides (e.g., consecutive nucleotides) of the nucleotide sequence of any of SEQ ID NOS: 1-4 (e.g., encoding a biologically active fragment of the dep1 polypeptide of any of SEQ ID NOS: 9-13); (c) a nucleotide sequence having at least about 60%, 70%, 75%, 80%, 85%, 90%, 95%, 97%, 98%, 99% or more sequence identity to the nucleotide sequence of (a) or (b); (d) a nucleotide sequence that hybridizes to the complete complement of the nucleotide sequence of (a) or (b) under stringent hybridization conditions; and (e) a nucleotide sequence that differs from the nucleotide sequence of any of (a) to (d) due to the degeneracy of the genetic code. In representative embodiments, the nucleotide sequence encodes a biologically active dep1 polypeptide that increases NUE in a plant.

In representative embodiments, the nucleotide sequence encodes the polypeptide of any of SEQ ID NOS: 9-13, or an equivalent polypeptide having substantial amino acid sequence identity or similarity with any of SEQ ID NOS: 9-13 (optionally, a biologically active equivalent that increases NUE). In representative embodiments, the nucleotide sequence encodes an equivalent (optionally, a biologically active equivalent) of the polypeptide of any of SEQ ID NOS: 9-13 and hybridizes to the complete complement of the nucleotide sequence of any of SEQ ID NO: 1-4 under stringent hybridization conditions.

In representative embodiments, the nucleotide sequence encodes the polypeptide of any of SEQ ID NOS: 9-13. According to this embodiment, the nucleotide sequence can comprise, consist essentially of, or consist of any of SEQ ID NOS: 1-4.

The dep1 polypeptides according to the present invention result in an increase in NUE and, optionally, a semi-dwarf phenotype in a plant. The dep1 polypeptides that “comprise” an indicated amino acid sequence or are encoded by a nucleic acid “comprising” a specified nucleotide sequence explicitly exclude DEP1 polypeptides and nucleotide sequences encoding the same, which do not result in an increase in NUE in a plant and/or a semi-dwarf phenotype.

### III. METHODS OF REDUCING THE AMOUNT AND/OR ACTIVITY OF A DEP1 POLYPEPTIDE IN A PLANT TO INCREASE NUE

The invention further encompasses a method of increasing NUE in a plant, the method comprising decreasing the amount and/or activity of a DEP1 polypeptide (e.g., native DEP1 polypeptide) in the plant, thereby resulting in an increase in NUE. In embodiments, the expression of a native DEP1 gene is reduced. In representative embodiments, the plant can be homozygous and/or heterozygous for the DEP1 allele. In embodiments of the invention, the plant is a transgenic plant comprising an isolated nucleic acid encoding a dep1 polypeptide, as discussed in more detail herein. The increase in NUE can be assessed with respect to any relevant control plant, e.g., a plant in which the amount and/or activity of a DEP1 polypeptide is not reduced according to the methods of the invention. The control plant is generally matched for species, variety, age, and the like and is subjected to the same growing conditions, e.g., temperature, soil, sunlight, pH, water, and the like. The selection of a suitable control plant is routine for those skilled in the art.

In representative embodiments, there is an increase in yield of the plant having a reduction in the amount and/or activity of a DEP1 polypeptide (including the yield of a plant product) as compared with a control plant at a given level of nitrogen (e.g., from the growing medium and/or in the form of nitrogen fertilizer). In embodiments of the invention, there is an increase in the ratio of a plant product (e.g., the grain dry mass) to above-ground nitrogen in the plant as compared with a control plant, for example, at a given level of nitrogen (e.g., from the growing medium and/or in the form of nitrogen fertilizer).

According to representative embodiments, the plant having a reduction in the amount and/or activity of a DEP1 polypeptide can be grown under conditions of standard, or even high, nitrogen conditions. In other exemplary embodiments, the plant is grown under low nitrogen conditions. The low nitrogen conditions can arise from the application of a reduced level of nitrogen fertilizer to the plant and/or growing the plant in a low nitrogen medium (e.g., soil, water, and the like). According to this embodiment, there is optionally an increase in yield of the plant (including the yield of a plant product) as compared with a control plant grown under the low nitrogen conditions. In embodiments of the invention, there is an increase in the ratio of a plant product (e.g., the grain dry mass) to above-ground nitrogen in the plant as compared with a control plant under the low nitrogen conditions.

In additional representative embodiments, the method comprises delivering an antibody and/or aptamer that specifically binds the DEP1 polypeptide and reduces the activity thereof. Such methods can further comprise introducing a nucleic acid into a plant that encodes the antibody and/or aptamer. The plant can be transiently or stably transformed with the nucleic acid encoding the antibody and/or aptamer. The nucleic acid can be introduced directly into the plant or a plant material (e.g., a plant cell or tissue such as a callus cell or tissue) and a plant regenerated therefrom, wherein the plant optionally comprises in its genome the nucleic acid encoding the antibody and/or aptamer.

In additional embodiments, the method of reducing the amount and/or activity of a DEP1 polypeptide comprises reducing the level of a nucleic acid (e.g., native nucleic acid) encoding the DEP1 polypeptide. According to representative embodiments, this method can comprise delivering an inhibitory polynucleotide (e.g., an antisense polynucleotide, an RNAi, a miRNA, an RNA aptamer and/or a ribozyme) to the plant to reduce the level of a nucleic acid encoding the DEP1 polypeptide. Such methods of “knocking down” the expression level of a native gene are well-known in the art. The method can further comprise introducing a nucleic acid into a plant that encodes the inhibitory polynucleotide. The plant can be transiently or stably transformed with the nucleic acid encoding the polynucleotide. The nucleic acid encoding the inhibitory polynucleotide can be introduced directly into the plant or into a plant material (e.g., a plant cell or tissue such as a callus cell or tissue) and a plant regenerated therefrom, wherein the plant optionally comprises in its genome the nucleic acid encoding the antibody and/or aptamer.

Numerous methods for reducing the level and/or expression of nucleic acids in vitro or in vivo are known. For example, the coding (and non-coding) sequences for a number of DEP1 polypeptides are known to those of skill in the art (see, e.g., SEQ ID NOS: 5-8 and U.S. Patent Application Publication 2011/0197305 A1). An inhibitory polynucleotide or nucleic acid encoding the same can be generated to any portion thereof in accordance with known techniques.

The term “antisense nucleotide sequence” or “antisense oligonucleotide” as used herein, refers to a nucleotide sequence that is complementary to a specified DNA or RNA sequence. Antisense oligonucleotides and nucleic acids that express the same can be made in accordance with conventional techniques. See, e.g., U.S. Pat. No. 5,023,243 to Tullis; U.S. Pat. No. 5,149,797 to Pederson et al. The antisense nucleotide sequence can be complementary to the entire nucleotide sequence encoding the polypeptide or a portion thereof of at least about 10, 20, 40, 50, 75, 100, 150, 200, 300, or 500 contiguous bases and will reduce the level of polypeptide production.

In additional embodiments, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of the polypeptide. As is known in the art, a higher degree of sequence similarity is generally required for short antisense nucleotide sequences, whereas a greater degree of mismatched bases will be tolerated by longer antisense nucleotide sequences. For example, hybridization of such nucleotide sequences can be carried out under conditions of reduced stringency, medium stringency or even stringent conditions (e.g., as described herein).

In other embodiments, antisense nucleotide sequences of the invention have at least about 70%, 80%, 90%, 95%, 97%, 98% or higher sequence similarity with the complement of the target sequence.

The length of the antisense polynucleotide (i.e., the number of nucleotides therein) is not critical as long as it binds selectively to the intended location and reduces transcription and/or translation of the target sequence, and can be determined in accordance with routine procedures. In general, the antisense nucleotide sequence will be from about eight, ten or twelve nucleotides in length up to about 20, 30, 50, 75 or 100 nucleotides, or longer, in length.

Triple helix base-pairing methods can also be employed to inhibit production of a DEP1 polypeptide. Triple helix pairing is believed to work by inhibiting the ability of the double helix to open sufficiently for the binding of polymerases, transcription factors, or regulatory molecules. Recent therapeutic advances using triplex DNA have been described in the literature (e.g., Gee et al., (1994) In: Huber et al., Molecular and Immunologic Approaches, Futura Publishing Co., Mt. Kisco, N.Y.).

Small Interference (si) RNA and short hairpin (sh) RNA, also known as RNA interference (RNAi) molecules, provides another approach for modulating the expression of a DEP1 polypeptide. The siRNA can be directed against polynucleotide sequences encoding the DEP1 polypeptides or any other sequence that results in modulation of the expression of the listed polypeptides.

siRNA is a mechanism of post-transcriptional gene silencing in which double-stranded RNA (dsRNA) corresponding to a coding sequence of interest is introduced into a cell or an organism, resulting in degradation of the corresponding mRNA. The mechanism by which siRNA achieves gene silencing has been reviewed in Sharp et al., Genes Dev. 15:485 (2001); and Hammond et al., Nature Rev. Gen. 2:110 (2001)). The siRNA effect persists for multiple cell divisions before gene expression is regained. siRNA is therefore a powerful method for making targeted knockouts or “knockdowns” at the RNA level. siRNA has proven successful in human cells, including human embryonic kidney and HeLa cells (see, e.g., Elbashir et al., Nature 411:494 (2001)). In one embodiment, silencing can be induced by enforcing endogenous expression of RNA hairpins (see Paddison et al., Proc. Natl. Acad. Sci. USA 99:1443 (2002)). In another embodiment, transfection of small (21-23 nt) dsRNA specifically inhibits nucleic acid expression (reviewed in Caplen, Trends Biotechnol. 20:49 (2002)).

siRNA technology utilizes standard molecular biology methods. dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in siRNA are available commercially, e.g., from New England Biolabs, Inc. Methods of transfection of dsRNA or plasmids engineered to make dsRNA are routine in the art.

shRNA is generally an RNA molecule that contains a sense strand, antisense strand, and a short loop sequence between the sense and antisense fragments. Due to the complementarity of the sense and antisense fragments in their sequence, such RNA molecules tend to form hairpin-shaped dsRNA. shRNA can be expressed in a cell, where they transported to the cytoplasm are processed by Dicer into siRNA.

MicroRNA (miRNA), single stranded RNA molecules of about 21-23 nucleotides in length, can be used in a similar fashion to siRNA to modulate gene expression (see U.S. Pat. No. 7,217,807).

Silencing effects similar to those produced by siRNA have been reported in mammalian cells with transfection of a mRNA-cDNA hybrid construct (Lin et al., Biochem. Biophys. Res. Commun. 281:639 (2001)), providing yet another strategy for silencing a coding sequence of interest.

The expression of a DEP1 polypeptide can also be inhibited using ribozymes. Ribozymes are RNA-protein complexes that cleave nucleic acids in a site-specific fashion. Ribozymes have specific catalytic domains that possess endonuclease activity (Kim et al., Proc. Natl. Acad. Sci. USA 84:8788 (1987); Gerlach et al., Nature 328:802 (1987); Forster and Symons, Cell 49:211 (1987)). For example, a large number of ribozymes accelerate phosphoester transfer reactions with a high degree of specificity, often cleaving only one of several phosphoesters in an oligonucleotide substrate (Michel and Westhof, J. Mol. Biol. 216:585 (1990); Reinhold-Hurek and Shub, Nature 357:173 (1992)). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, Nature 338:217 (1989)). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., Proc. Natl. Acad. Sci. USA 88:10591 (1991); Sarver et al., Science 247:1222 (1990); Sioud et al., J. Mol. Biol. 223:831 (1992)).

An inhibitory polynucleotide sequence can be constructed using chemical synthesis and enzymatic ligation reactions by procedures known in the art. For example, an inhibitory polynucleotide sequence can be chemically synthesized using naturally occurring nucleotides or various modified nucleotides designed to increase the biological stability of the molecules or to increase the physical stability of the duplex formed between the inhibitory polynucleotide and target nucleotide sequence, e.g., phosphorothioate derivatives and acridine substituted nucleotides can be used. Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The inhibitory polynucleotide sequences of the invention further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every other one of the intemucleotide bridging phosphate residues can be modified as described. In another non-limiting example, the inhibitory polynucleotide sequence is a nucleotide sequence in which one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sci. USA 85:5011 (1988)).

When delivering a nucleic acid encoding an inhibitory polynucleotide, antibody and/or aptamer to plant or plant material (e.g., plant cell) according to a method of the invention, the nucleic acid can optionally be operably associated with a tissue-specific or tissue-preferred promoter, e.g., a root-specific or -preferred, leaf-specific or -preferred, inflorescence-specific or -preferred, or meristem-specific or -preferred promoters.

In another embodiment of the invention, reducing the expression and/or activity of a DEP1 polypeptide comprises decreasing the activity of the polypeptide. Polypeptide activity can be modulated by interaction with an antibody (including antibody fragments). The antibody can bind to the DEP1 polypeptide or to any other polypeptide of interest, as long as the binding between the antibody and the target polypeptide results in modulation of the activity of a DEP1 polypeptide.

The term “antibody” or “antibodies” as used herein refers to all types of immunoglobulins, including IgG, IgM, IgA, IgD, and IgE, and includes antibody fragments. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, Fab′, F(ab′)₂, and Fv fragments; domain antibodies, diabodies; vaccibodies, linear antibodies; single-chain antibody molecules; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254:1275 (1989)).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, Nature 265:495 (1975). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse, Science 246:1275 (1989).

Antibodies specific to the target polypeptide can also be obtained by phage display techniques known in the art.

Various immunoassays can be used for screening to identify antibodies having the desired specificity for a DEP1 polypeptide. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes on the polypeptides or peptides of this invention can be used as well as a competitive binding assay.

Antibodies can be conjugated to a solid support (e.g., beads, plates, slides or wells formed from materials such as latex or polystyrene) in accordance with known techniques. Antibodies can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

In one embodiment, the activity of DEP1 is inhibited using an aptamer, which term includes peptide aptamers and RNA aptamers. RNA aptamers, which are small structured single-stranded RNAs, have emerged as viable alternatives to small-molecule and antibody-based therapy (Que-Gewirth et al., Gene Ther. 14:283 (2007); Ireson et al., Mol. Cancer Ther. 5:2957 (2006)). RNA aptamers specifically bind target proteins with high affinity, are quite stable, lack immunogenicity, and elicit biological responses. Aptamers can be evolved by means of an iterative selection method called SELEX (systematic evolution of ligands by exponential enrichment) to specifically recognize and tightly bind their targets by means of well-defined complementary three-dimensional structures.

RNA aptamers represent an emerging class of therapeutic agents (Que-Gewirth et al., Gene Ther. 14:283 (2007); Ireson et al., Mol. Cancer Ther. 5:2957 (2006)). They are generally relatively short (12-30 nucleotide) single-stranded RNA oligonucleotides that assume a stable three-dimensional shape to tightly and specifically bind selected protein targets to elicit a biological response. Like antibodies, aptamers possess binding affinities in the low nanomolar to picomolar range. In addition, aptamers are heat stable, lack immunogenicity, and possess minimal interbatch variability. Chemical modifications, such as amino or fluoro substitutions at the 2′ position of pyrimidines, may reduce degradation by nucleases. The biodistribution and clearance of aptamers can also be altered by chemical addition of moieties such as polyethylene glycol and cholesterol. Further, SELEX allows selection from libraries consisting of up to 10¹⁵ ligands to generate high-affinity oligonucleotide ligands to purified biochemical targets.

Peptide aptamers resemble single chain antibodies, but because they are often selected in vivo selection, they may be more likely to be stably expressed and correctly folded and to interact with their targets in an intracellular context. There are numerous reports of successful regulation of cellular functions using peptide aptamers. For example, an aptamer that binds to the active site of the cell cycle regulator, cdk2, was isolated by screening a combinatorial peptide library in yeast dihybrid assays (Colas et al. (1996) Nature 380:548-550). The aptamer blocks cdk2/cyclin E kinase activity in vitro and, when expressed in vivo, retards cell division. An aptamer that interacts with the dimerization domain of cell cycle-associated transcription factor, E2F, also interferes with cell cycle progression in animal cells (Fabbrizio et al, 1999 Oncogene 18:4357-4363). Aptamers have also been expressed in flies to study the specific roles of cdk1 and cdk2 during Drosophila organogenesis (Kolonin and Finley, 1998 PNAS USA 95: 14266-14271). They have been used to distinguish between and selectively inactivate allelic variants of Ras and to inhibit Rho GTP exchange factors (Schmidt et al, 2002 FEBS Letters 523:35-42) as well as interfere with the EGF signaling pathway by binding to the downstream transcription factor—Stat3 (Nagel-Wolfrum, Buerger et al., 2004 Mol. Cancer Res. 2: 170-182).

In representative embodiments in which a nucleic acid is introduced in a plant to reduce the amount and/or activity of a DEP1 polypeptide, the method comprises: (a) introducing the nucleic acid (e.g., isolated nucleic acid) encoding an inhibitory polynucleotide, antibody and/or aptamer into a plant cell (including a callus cell) to produce a transgenic plant cell; and (b) regenerating a transgenic plant from the transgenic plant cell of (a), optionally wherein the transgenic plant comprises in its genome the nucleic acid encoding an inhibitory polynucleotide, antibody and/or aptamer and has increased NUE as compared with a control plant (e.g., expresses the inhibitory polynucleotide, antibody and/or aptamer in an amount effective to increase NUE in the plant).

In representative embodiments, the method further comprises selecting from a plurality of the transgenic plants of (b) a transgenic plant having increased NUE.

The invention also contemplates the production of progeny plants that comprise the nucleic acid encoding an inhibitory polynucleotide, antibody and/or aptamer. In embodiments of the invention, the method further comprises obtaining a progeny plant derived from the transgenic plant (e.g., by sexual reproduction or vegetative propagation), optionally wherein the progeny plant comprises in its genome the isolated nucleic acid encoding an inhibitory polynucleotide, antibody and/or aptamer and has increased NUE as compared with a control plant (e.g., expresses the inhibitory polynucleotide, antibody and/or aptamer in an amount effective to increase NUE in the plant).

To illustrate, in one embodiment, the invention provides a method of producing a progeny plant, the method comprising (a) crossing the transgenic plant comprising the nucleic acid encoding an inhibitory polynucleotide, antibody and/or aptamer with itself or another plant to produce seed comprising the nucleic acid encoding an inhibitory polynucleotide, antibody and/or aptamer; and (b) growing a progeny plant from the seed to produce a transgenic plant, optionally wherein the progeny plant comprises in its genome the isolated nucleic acid encoding an inhibitory polynucleotide, antibody and/or aptamer and has increased NUE as compared with a control plant (e.g., expresses the inhibitory polynucleotide, antibody and/or aptamer in an amount effective to increase NUE in the plant). In additional embodiments, the method can further comprise (c) crossing the progeny plant with itself or another plant and (d) repeating steps (b) and (c) for an additional 0-7 (e.g., 0, 1, 2, 3, 4, 5, 6 or 7 and any range thereof) generations to produce a plant, optionally wherein the plant comprises in its genome the isolated nucleic acid encoding an inhibitory polynucleotide, antibody and/or aptamer and has increased NUE as compared with a control plant (e.g., expresses the inhibitory polynucleotide, antibody and/or aptamer in an amount effective to increase NUE in the plant).

In another embodiment, the method of reducing the activity of a DEP1 polypeptide comprises delivering to the plant a compound that reduces the activity of the DEP1 polypeptide, the compound administered in an amount effective to modulate the reduce the activity of the DEP1 polypeptide. The compound can interact directly with the DEP1 polypeptide to decrease the activity thereof. Alternatively, the compound can interact with any other polypeptide, nucleic acid or other molecule if such interaction results in a decrease of the activity of the DEP1 polypeptide.

The term “compound” as used herein is intended to be interpreted broadly and encompasses organic and inorganic molecules. Organic compounds include, but are not limited to, small molecules, polypeptides, lipids, carbohydrates, coenzymes, aptamers, and nucleic acid molecules (e.g., gene delivery vectors, antisense oligonucleotides, siRNA, all as described above).

Polypeptides include, but are not limited to, antibodies (described in more detail above) and enzymes. Nucleic acids include, but are not limited to, DNA, RNA and DNA-RNA chimeric molecules. Suitable RNA molecules include siRNA, antisense RNA molecules and ribozymes (all of which are described in more detail above). The nucleic acid can further encode any polypeptide such that administration of the nucleic acid and production of the polypeptide results in a decrease of the activity of a DEP1 polypeptide.

### IV. EXPRESSION CASSETTES

In representative embodiments, the nucleic acids, polynucleotides and nucleotide sequences of the invention are comprised within an expression cassette and are in operable association with a heterologous promoter. In some embodiments, the expression cassette comprises a nucleic acid encoding a dep1 polypeptide of the invention operably associated with a promoter. In embodiments, the nucleic acid encoding the dep1 polypeptide is operably associated with the native promoter. In particular embodiments, the nucleic acid encoding the dep1 polypeptide is operably associated with a heterologous promoter.

In additional embodiments, the expression cassette comprises a nucleic acid encoding an inhibitory polynucleotide, antibody and/or aptamer for reducing the amount and/or activity of a DEP1 polypeptide operably associated with a heterologous promoter.

The heterologous promoter can be any suitable promoter known in the art (including bacterial, yeast, fungal, insect, mammalian, and plant promoters). In particular embodiments, the promoter is a promoter for expression in plants. The selection of promoters suitable for use with the present invention can be made among many different types of promoters. Thus, the choice of promoter depends upon several factors, including, but not limited to, cell- or tissue-specific expression, desired expression level, efficiency, inducibility and/or selectability. For example, where expression in a specific tissue or organ is desired in addition to inducibility, a tissue-specific or tissue-preferred promoter can be used (e.g., a root specific or preferred promoter). In contrast, where expression in response to a stimulus is desired a promoter inducible by other stimuli or chemicals can be used. Where continuous expression is desired throughout the cells of a plant, a constitutive promoter can be chosen.

Non-limiting examples of constitutive promoters include cestrum virus promoter (cmp) (U.S. Pat. No. 7,166,770), an actin promoter (e.g., the rice actin 1 promoter; Wang et al. (1992) Mol. Cell. Biol. 12:3399-3406; as well as U.S. Pat. No. 5,641,876), Cauliflower Mosaic Virus (CaMV) 35S promoter (Odell et al. (1985) Nature 313:810-812), CaMV 19S promoter (Lawton et al. (1987) Plant Mol. Biol. 9:315-324), an opine synthetase promoter (e.g., nos, mas, ocs, etc.; (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and a ubiquitin promoter.

Some non-limiting examples of tissue-specific promoters for use with the present invention include those derived from genes encoding seed storage proteins (e.g., β-conglycinin, cruciferin, napin phaseolin, etc.), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, the promoters associated with these tissue-specific nucleic acids can be used in the present invention.

Additional examples of tissue-specific promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants, Hollaender ed., Plenum Press 1983; and Poulsen et al. (1986) Mol. Gen. Genet. 205:193-200)), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. Other promoters useful with the present invention include but are not limited to the S-E9 small subunit RuBP carboxylase promoter and the Kunitz trypsin inhibitor gene promoter (Kti3).

Other tissue-specific or tissue-preferred promoters include inflorescence-specific or preferred and meristem-specific or -preferred promoters.

In some embodiments, inducible promoters can be used with the present invention. Examples of inducible promoters useable with the present invention include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), the glyceraldehyde-3-phosphate dehydrogenase promoter (Kohler et al. (1995) Plant Mol. Biol. 29:1293-1298; Martinez et at (1989) J. Mol. Biol. 208:551-565; and Quigley et al. (1989) J. Mol. Evol. 29:412-421) the benzene sulphonamide-inducible promoters (U.S. Pat. No. 5,364,780) and the glutathione S-transferase promoters. Likewise, one can use any appropriate inducible promoter described in Gatz (1996) Current Opinion Biotechnol. 7:168-172 and Gatz (1997) Annu. Rev. Plant Physiol. Plant Mol. Biol. 48:89-108.

Other suitable promoters include promoters from viruses that infect the host plant including, but not limited to, promoters isolated from Dasheen mosaic virus, Chlorella virus (e.g., the Chlorella virus adenine methyltransferase promoter; Mitra et al., (1994) Plant Molecular Biology 26:85), tomato spotted wilt virus, tobacco rattle virus, tobacco necrosis virus, tobacco ring spot virus, tomato ring spot virus, cucumber mosaic virus, peanut stump virus, alfalfa mosaic virus, and the like.

The expression cassettes of the invention may optionally further comprise a transcriptional termination sequence. Any suitable termination sequence known in the art may be used in accordance with the present invention. The termination region may be native with the transcriptional initiation region, may be native with the nucleotide sequence of interest, or may be derived from another source. Convenient termination regions are available from the Ti-plasmid of A. tumefaciens, such as the octopine synthetase and nopaline synthetase termination regions. See also, Guerineau et al., Mol. Gen. Genet. 262, 141 (1991); Proudfoot, Cell 64, 671 (1991); Sanfacon et al., Genes Dev. 5,141 (1991); Mogen et al., Plant Cell 2, 1261 (1990); Munroe et al., Gene 91, 151 (1990); Ballas et al., Nucleic Acids Res. 17, 7891 (1989); and Joshi et al., Nucleic Acids Res. 15, 9627 (1987). Additional exemplary termination sequences are the pea RubP carboxylase small subunit termination sequence and the Cauliflower Mosaic Virus 35S termination sequence. Other suitable termination sequences will be apparent to those skilled in the art.

Further, in particular embodiments, the nucleic acid, polynucleotide or nucleotide sequence of interest is operably associated with a translational start site. The translational start site can be the native translational start site or any other suitable translational start codon.

In illustrative embodiments, the expression cassette includes in the 5′ to 3′ direction of transcription, a promoter, a nucleotide sequence of interest (e.g., a nucleotide sequence encoding a dep1 polypeptide, an inhibitory polynucleotide, an antibody and/or an aptamer), and a transcriptional and translational termination region functional in plants.

Those skilled in the art will understand that the expression cassettes of the invention can further comprise enhancer elements and/or tissue preferred elements in combination with the promoter.

Further, in some embodiments, it is advantageous for the expression cassette to comprise a selectable marker gene for the selection of transformed cells. Selectable marker genes include genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Selectable marker genes that can be used according to the present invention further include, but are not limited to, genes encoding: neomycin phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Perl et al., BioTechnology 11, 715 (1993)); the bar gene (Toki et al., Plant Physiol. 100, 1503 (1992); Meagher et al., Crop Sci. 36, 1367 (1996)); tryptophane decarboxylase (Goddijn et al., Plant Mol. Biol. 22, 907 (1993)); neomycin phosphotransferase (NEO; Southern et al., J. Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR; Kwok et al., Proc. Natl. Acad. Sci. USA 83, 4552 (1986)); phosphinothricin acetyltransferase (DeBlock et al., EMBO J. 6, 2513 (1987)); 2,2-dichloropropionic acid dehalogenase (Buchanan-Wollatron et al., J. Cell. Biochem. 13D, 330 (1989)); acetohydroxyacid synthase (U.S. Pat. No. 4,761,373 to Anderson et al.; Haughn et al., Mol. Gen. Genet. 221, 266 (1988)); 5-enolpyruvyl-shikimate-phosphate synthase (aroA; Comai et al., Nature 317, 741 (1985)); haloarylnitrilase (WO 87/04181 to Stalker et al.); acetyl-coenzyme A carboxylase (Parker et al., Plant Physiol. 92, 1220 (1990)); dihydropteroate synthase (sulI; Guerineau et al., Plant Mol. Biol. 15, 127 (1990)); and 32 kDa photosystem II polypeptide (psbA; Hirschberg et al., Science 222, 1346 (1983)).

Also included are genes encoding resistance to: chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5, 103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); and spectinomycin (Bretagne-Sagnard et al., Transgenic Res. 5, 131 (1996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4-D (Streber et al., Bio/Technology 7, 811 (1989)); phosphinothricin (DeBlock et al., EMBO J. 6, 2513 (1987)); spectinomycin (Bretagne-Sagnard and Chupeau, Transgenic Research 5, 131 (1996)).

Other selectable marker genes include the pat gene (for bialaphos and phosphinothricin resistance), the ALS gene for imidazolinone resistance, the HPH or HYG gene for hygromycin resistance, the Hm1 gene for resistance to the Hc-toxin, and other selective agents used routinely and known to one of ordinary skill in the art. See generally, Yarranton, Curr. Opin. Biotech. 3, 506 (1992); Chistopherson et al., Proc. Natl. Acad. Sci. USA 89, 6314 (1992); Yao et al., Cell 71, 63 (1992); Reznikoff, Mol. Microbiol. 6, 2419 (1992); BARKLEY ET AL., THE OPERON 177-220 (1980); Hu et al., Cell 48, 555 (1987); Brown et al., Cell 49, 603 (1987); Figge et al., Cell 52, 713 (1988); Deuschle et al., Proc. Natl. Acad. Sci. USA 86, 5400 (1989); Fuerst et al., Proc. Natl. Acad. Sci. USA 86, 2549 (1989); Deuschle et al., Science 248, 480 (1990); Labow et al., Mol. Cell. Biol. 10, 3343 (1990); Zambretti et al., Proc. Natl. Acad. Sci. USA 89, 3952 (1992); Baim et al., Proc. Natl. Acad. Sci. USA 88, 5072 (1991); Wyborski et al., Nuc. Acids Res. 19, 4647 (1991); Hillenand-Wissman, Topics in Mol. And Struc. Biol. 10, 143 (1989); Degenkolb et al., Antimicrob. Agents Chemother. 35, 1591 (1991); Kleinschnidt et al., Biochemistry 27, 1094 (1988); Gatz et al., Plant J. 2, 397 (1992); Gossen et al., Proc. Natl. Acad. Sci. USA 89, 5547 (1992); Oliva et al., Antimicrob. Agents Chemother. 36, 913 (1992); HLAVKA ET AL., HANDBOOK OF EXPERIMENTAL PHARMACOLOGY 78 (1985); and Gill et al., Nature 334, 721 (1988).

The nucleotide sequence of interest can additionally be operably linked to a sequence that encodes a transit peptide that directs expression of an encoded polypeptide of interest to a particular cellular compartment. Transit peptides that target protein accumulation in higher plant cells to the chloroplast, mitochondrion, vacuole, nucleus, and the endoplasmic reticulum are known in the art. For example, transit peptides that target proteins to the endoplasmic reticulum are desirable for correct processing of secreted proteins. Targeting protein expression to the chloroplast (for example, using the transit peptide from the RubP carboxylase small subunit gene) has been shown to result in the accumulation of very high concentrations of recombinant protein in this organelle. The pea RubP carboxylase small subunit transit peptide sequence has been used to express and target mammalian genes in plants (U.S. Pat. Nos. 5,717,084 and 5,728,925 to Herrera-Estrella et al.). Alternatively, mammalian transit peptides can be used to target recombinant protein expression, for example, to the mitochondrion and endoplasmic reticulum. It has been demonstrated that plant cells recognize mammalian transit peptides that target endoplasmic reticulum (U.S. Pat. Nos. 5,202,422 and 5,639,947 to Hiatt et al.).

Further, the expression cassette can comprise a 5′ leader sequence that acts to enhance expression (transcription, post-transcriptional processing and/or translation) of an operably associated nucleotide sequence of interest. Leader sequences are known in the art and include sequences from: picornavirus leaders, e.g., EMCV leader (Encephalomyocarditis 5′ noncoding region; Elroy-Stein et al., Proc. Natl. Acad. Sci USA, 86, 6126 (1989)).; potyvirus leaders, e.g., TEV leader (Tobacco Etch Virus; Allison et al., Virology, 154, 9 (1986)); human immunoglobulin heavy-chain binding protein (BiP; Macajak and Sarnow, Nature 353, 90 (1991)); untranslated leader from the coat protein mRNA of alfalfa mosaic virus (AMV RNA 4; Jobling and Gehrke, Nature 325, 622 (1987)); tobacco mosaic virus leader (TMV; Gallie, MOLECULAR BIOLOGY OF RNA, 237-56 (1989)); and maize chlorotic mottle virus leader (MCMV; Lommel et al., Virology 81, 382 (1991)). See also, Della-Cioppa et al., Plant Physiology 84, 965 (1987).

The nucleotide sequence of interest in the expression cassette can be any nucleotide sequence(s) of interest for practicing the present invention and can be, for example, a nucleotide sequence that encodes a dep1 polypeptide or a complement thereof (e.g., a complete complement). Other suitable nucleotide sequences of interest include without limitation those that encode an inhibitory polynucleotide, an antibody and/or an aptamer to reduce Dep1 expression in a plant, plant part or plant cell.

The expression cassette can further comprise a heterologous nucleotide sequence encoding a reporter polypeptide (e.g., an enzyme), including but not limited to Green Fluorescent Protein, β-galactosidase, luciferase, alkaline phosphatase, the GUS gene encoding β-glucuronidase, and chloramphenicol acetyltransferase.

Where appropriate, the heterologous nucleic acids may be optimized for increased expression in a transformed plant, e.g., by using plant preferred codons. Methods for synthetic optimization of nucleic acid sequences are available in the art. The nucleotide sequence can be optimized for expression in a particular host plant or alternatively can be modified for optimal expression in monocots. See, e.g., EP 0 359 472, EP 0 385 962, WO 91/16432; Perlak et al., Proc. Natl. Acad. Sci. USA 88, 3324 (1991), and Murray et al., Nuc. Acids Res. 17, 477 (1989), and the like. Plant preferred codons can be determined from the codons of highest frequency in the proteins expressed in that plant.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The invention further provides vectors comprising the nucleic acids and expression cassettes of the invention, including expression vectors, transformation vectors and vectors for replicating and/or manipulating the nucleotide sequences in the laboratory. The vector can be a plant vector, animal (e.g., insect or mammalian) vector, bacterial vector, yeast vector or fungal vector. Generally, according to the present invention, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses.

### V. TRANSGENIC PLANTS, PLANT PARTS AND PLANT CELLS

The invention also provides transgenic plants, plant parts and plant cells comprising the nucleic acids, expression cassettes and vectors of the invention.

Accordingly, as one aspect the invention provides a cell comprising a nucleic acid, expression cassette, or vector of the invention. The cell can be transiently or stably transformed with the nucleic acid, expression cassette or vector. Further, the cell can be a cultured cell, a cell obtained from a plant, plant part, or plant tissue, or a cell in situ in a plant, plant part or plant tissue. Cells can be from any suitable species, including plant (e.g. rice), bacterial, yeast, insect and/or mammalian cells. In representative embodiments, the cell is a plant cell or bacterial cell.

The invention also provides a plant part (including a plant tissue culture) comprising a nucleic acid, expression cassette, or vector of the invention. The plant part can be transiently or stably transformed with the nucleic acid, expression cassette or vector. Further, the plant part can be in culture, can be a plant part obtained from a plant, or a plant part in situ. In representative embodiments, the plant part comprises a cell of the invention (e.g., as described in the preceding paragraph).

Seed comprising the nucleic acid, expression cassette, or vector of the invention are also provided. Optionally, the nucleic acid, expression cassette or vector is stably incorporated into the genome of the seed.

The invention also contemplates a transgenic plant comprising a nucleic acid, expression cassette, or vector of the invention. The plant can be transiently or stably transformed with the nucleic acid, expression cassette or vector. In representative embodiments, the plant comprises a cell or plant part of the invention (as described above). In representative embodiments, wherein the nucleic acid, expression cassette or vector encodes a dep1 polypeptide the transgenic plant has increased NUE. In representative embodiments, the nucleic acid, expression cassette or vector encodes an inhibitory polynucleotide, an antibody and/or aptamer that reduces the amount and/or activity of a Dep1 polypeptide, and the transgenic plant has increased NUE.

Still further, the invention encompasses a crop comprising a plurality of the transgenic plants of the invention, as described herein. Nonlimiting examples of the types of crops comprising a plurality of transgenic plants of the invention include an agricultural field, a golf course, a residential lawn or garden, a public lawn or garden, a road side planting, an orchard, and/or a recreational field (e.g., a cultivated area comprising a plurality of the transgenic plants of the invention).

Products harvested from the plants of the invention are also provided. Nonlimiting examples of a harvested product include a seed, a leaf, a stem, a shoot, a fruit, flower, root, biomass (e.g., for biofuel production) and/or extract.

In some embodiments, a processed product produced from the harvested product is provided. Nonlimiting examples of a processed product include a protein (e.g., a recombinant protein), an extract, a medicinal product (e.g., artemicin as an antimalarial agent), a fiber or woven textile, a fragrance, dried fruit, a biofuel (e.g., ethanol), a tobacco product (e.g., cured tobacco, cigarettes, chewing tobacco, cigars, and the like), an oil (e.g., sunflower oil, corn oil, canola oil, and the like), a nut or seed butter, a flour or meal (e.g., wheat or rice flour, corn meal) and/or any other animal feed (e.g., soy, maize, barley, rice, alfalfa) and/or human food product (e.g., a processed wheat, maize, rice or soy food product).

### VI. METHODS OF INTRODUCING NUCLEIC ACIDS

The invention also provides methods of delivering a nucleic acid, expression cassette or vector of the invention to a target plant or plant cell (including callus cells or protoplasts), plant part, seed, plant tissue (including callus), and the like. The invention further comprises host plants, cells, plant parts, seed or tissue culture (including callus) transiently or stably transformed with the nucleic acids, expression cassettes or vectors of the invention.

The invention provides methods of introducing a dep1 polypeptide into a plant material, e.g., a plant, plant part (including callus) or plant cell. The invention also provides a method of introducing an inhibitory polynucleotide (or a nucleic acid encoding the same) or a nucleic acid encoding an antibody and/or aptamer that reduces the amount and/or activity of DEP1 into a plant material, e.g., a plant, plant part or plant cell. In representative embodiments, the method comprises transforming a plant cell with a nucleic acid, expression cassette, or vector of the invention encoding the dep1 polypeptide, the inhibitory polynucleotide, antibody and/or aptamer to produce a transformed plant cell, and regenerating a stably transformed transgenic plant from the transformed plant cell.

The invention further encompasses transgenic plants (and progeny thereof), plant parts, and plant cells produced by the methods of the invention.

Also provided by the invention are seed produced from the inventive transgenic plants. Optionally, the seed comprise a nucleic acid, expression cassette or vector of the invention stably incorporated into the genome.

Methods of introducing nucleic acids, transiently or stably, into plants, plant tissues, cells, protoplasts, seed, callus and the like are known in the art. Stably transformed nucleic acids can be incorporated into the genome. Exemplary transformation methods include biological methods using viruses and bacteria (e.g., Agrobacterium), physicochemical methods such as electroporation, floral dip methods, ballistic bombardment, microinjection, and the like. Other transformation technology includes the whiskers technology that is based on mineral fibers (see e.g., U.S. Pat. Nos. 5,302,523 and 5,464,765) and pollen tube transformation.

Other exemplary transformation methods include, without limitation, calcium-phosphate-mediated transformation, cyclodextrin-mediated transformation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some particular embodiments, the method of introducing into a plant, plant part, plant tissue, plant cell, protoplast, seed, callus and the like comprises bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or a combination thereof.

In one form of direct transformation, the vector is microinjected directly into plant cells by use of micropipettes to mechanically transfer the recombinant DNA (Crossway, Mol. Gen. Genetics 202: 179 (1985)).

In another protocol, the genetic material is transferred into the plant cell using polyethylene glycol (Krens, et al. Nature 296, 72 (1982)).

In still another method, protoplasts are fused with minicells, cells, lysosomes, or other fusible lipid-surfaced bodies that contain the nucleotide sequence to be transferred to the plant (Fraley, et al., Proc. Natl. Acad. Sci. USA 79, 1859 (1982)).

Nucleic acids may also be introduced into the plant cells by electroporation (Fromm et al., Proc. Natl. Acad. Sci. USA 82, 5824 (1985)). In this technique, plant protoplasts are electroporated in the presence of nucleic acids comprising the expression cassette. Electrical impulses of high field strength reversibly permeabilize biomembranes allowing the introduction of the nucleic acid. Electroporated plant protoplasts reform the cell wall, divide and regenerate. One advantage of electroporation is that large pieces of DNA, including artificial chromosomes, can be transformed by this method.

Ballistic transformation typically comprises the steps of: (a) providing a plant material as a target; (b) propelling a microprojectile carrying the heterologous nucleotide sequence at the plant target at a velocity sufficient to pierce the walls of the cells within the target and to deposit the nucleotide sequence within a cell of the target to thereby provide a transformed target. The method can further include the step of culturing the transformed target with a selection agent and, optionally, regeneration of a transformed plant. As noted below, the technique may be carried out with the nucleotide sequence as a precipitate (wet or freeze-dried) alone, in place of the aqueous solution containing the nucleotide sequence.

Any ballistic cell transformation apparatus can be used in practicing the present invention. Exemplary apparatus are disclosed by Sandford et al. (Particulate Science and Technology 5, 27 (1988)), Klein et al. (Nature 327, 70 (1987)), and in EP 0 270 356. Such apparatus have been used to transform maize cells (Klein et al., Proc. Natl. Acad. Sci. USA 85, 4305 (1988)), soybean callus (Christou et al., Plant Physiol. 87, 671 (1988)), McCabe et al., BioTechnology 6, 923 (1988), yeast mitochondria (Johnston et al., Science 240, 1538 (1988)), and Chlamydomonas chloroplasts (Boynton et al., Science 240, 1534 (1988)).

Alternately, an apparatus configured as described by Klein et al. (Nature 70, 327 (1987)) may be utilized. This apparatus comprises a bombardment chamber, which is divided into two separate compartments by an adjustable-height stopping plate. An acceleration tube is mounted on top of the bombardment chamber. A macroprojectile is propelled down the acceleration tube at the stopping plate by a gunpowder charge. The stopping plate has a borehole formed therein, which is smaller in diameter than the microprojectile. The macroprojectile carries the microprojectile(s), and the macroprojectile is aimed and fired at the borehole. When the macroprojectile is stopped by the stopping plate, the microprojectile(s) is propelled through the borehole. The target is positioned in the bombardment chamber so that a microprojectile(s) propelled through the bore hole penetrates the cell walls of the cells in the target and deposit the nucleotide sequence of interest carried thereon in the cells of the target. The bombardment chamber is partially evacuated prior to use to prevent atmospheric drag from unduly slowing the microprojectiles. The chamber is only partially evacuated so that the target tissue is not desiccated during bombardment. A vacuum of between about 400 to about 800 millimeters of mercury is suitable.

In alternate embodiments, ballistic transformation is achieved without use of microprojectiles. For example, an aqueous solution containing the nucleotide sequence of interest as a precipitate may be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described above). Other approaches include placing the nucleic acid precipitate itself (“wet” precipitate) or a freeze-dried nucleotide precipitate directly on the plate-contact end of the macroprojectile without a microprojectile. In the absence of a microprojectile, it is believed that the nucleotide sequence must either be propelled at the tissue target at a greater velocity than that needed if carried by a microprojectile, or the nucleotide sequenced caused to travel a shorter distance to the target (or both).

It particular embodiments, the nucleotide sequence is delivered by a microprojectile. The microprojectile can be formed from any material having sufficient density and cohesiveness to be propelled through the cell wall, given the particle's velocity and the distance the particle must travel. Non-limiting examples of materials for making microprojectiles include metal, glass, silica, ice, polyethylene, polypropylene, polycarbonate, and carbon compounds (e.g., graphite, diamond). Non-limiting examples of suitable metals include tungsten, gold, and iridium. The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles may enhance their carrying capacity.

The nucleotide sequence may be immobilized on the particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356 (column 8).

Alternatively, plants may be transformed using Agrobacterium tumefaciens or Agrobacterium rhizogenes. Agrobacterium-mediated nucleic acid transfer exploits the natural ability of A. tumefaciens and A. rhizogenes to transfer DNA into plant chromosomes. Agrobacterium is a plant pathogen that transfers a set of genes encoded in a region called T-DNA of the Ti and Ri plasmids of A. tumefaciens and A. rhizogenes, respectively, into plant cells. The typical result of transfer of the Ti plasmid is a tumorous growth called a crown gall in which the T-DNA is stably integrated into a host chromosome. Integration of the Ri plasmid into the host chromosomal DNA results in a condition known as “hairy root disease”. The ability to cause disease in the host plant can be removed by deletion of the genes in the T-DNA without loss of DNA transfer and integration. The DNA to be transferred is attached to border sequences that define the end points of an integrated T-DNA.

Transfer by means of engineered Agrobacterium strains has become routine for many dicotyledonous plants. Some difficulty has been experienced, however, in using Agrobacterium to transform monocotyledonous plants, in particular, cereal plants. However, Agrobacterium mediated transformation has been achieved in several monocot species, including cereal species such as rye, maize (Rhodes et al., Science 240, 204 (1988)), and rice (Hiei et al., (1994) Plant J. 6:271).

While the following discussion will focus on using A. tumefaciens to achieve gene transfer in plants, those skilled in the art will appreciate that this discussion also applies to A. rhizogenes. Transformation using A. rhizogenes has developed analogously to that of A. tumefaciens and has been successfully utilized to transform, for example, alfalfa, Solanum nigrum L., and poplar (U.S. Pat. No. 5,777,200 to Ryals et al.). As described by U.S. Pat. No. 5,773,693 to Burgess et al., it is preferable to use a disarmed A. tumefaciens strain (as described below), however, the wild-type A. rhizogenes may be employed. An illustrative strain of A. rhizogenes is strain 15834.

In particular protocols, the Agrobacterium strain is modified to contain the nucleotide sequences to be transferred to the plant. The nucleotide sequence to be transferred is incorporated into the T-region and is typically flanked by at least one T-DNA border sequence, optionally two T-DNA border sequences. A variety of Agrobacterium strains are known in the art particularly, and can be used in the methods of the invention. See, e.g., Hooykaas, Plant Mol. Biol. 13, 327 (1989); Smith et al., Crop Science 35, 301 (1995); Chilton, Proc. Natl. Acad. Sci. USA 90, 3119 (1993); Mollony et al., Monograph Theor. Appl. Genet NY 19, 148 (1993); Ishida et al., Nature Biotechnol. 14, 745 (1996); and Komari et al., The Plant Journal 10, 165 (1996).

In addition to the T-region, the Ti (or Ri) plasmid contains a vir region. The vir region is important for efficient transformation, and appears to be species-specific.

Two exemplary classes of recombinant Ti and Ri plasmid vector systems are commonly used in the art. In one class, called “cointegrate,” the shuttle vector containing the gene of interest is inserted by genetic recombination into a non-oncogenic Ti plasmid that contains both the cis-acting and trans-acting elements required for plant transformation as, for example, in the PMLJ1 shuttle vector of DeBlock et al., EMBO J. 3, 1681 (1984), and the non-oncogenic Ti plasmid pGV2850 described by Zambryski et al., EMBOJ 2, 2143 (1983). In the second class or “binary” system, the gene of interest is inserted into a shuttle vector containing the cis-acting elements required for plant transformation. The other necessary functions are provided in trans by the non-oncogenic Ti plasmid as exemplified by the pBIN19 shuttle vector described by Bevan, Nucleic Acids Research 12, 8711 (1984), and the non-oncogenic Ti plasmid PAL4404 described by Hoekma, et al., Nature 303, 179 (1983).

Binary vector systems have been developed where the manipulated disarmed T-DNA carrying the heterologous nucleotide sequence of interest and the vir functions are present on separate plasmids. In this manner, a modified T-DNA region comprising foreign DNA (the nucleic acid to be transferred) is constructed in a small plasmid that replicates in E. coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. Such binary vectors are useful in the practice of the present invention.

In particular embodiments of the invention, super-binary vectors are employed. See, e.g., U.S. Pat. No. 5,591,615 and EP 0 604 662. Such a super-binary vector has been constructed containing a DNA region originating from the hypervirulence region of the Ti plasmid pTiBo542 (Jin et al., J. Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J Bacteriol. 166, 88 (1986); Jin et al., J. Bacteriol. 169, 4417 (1987); Komari, Plant Science 60, 223 (1987); ATCC Accession No. 37394.

Exemplary super-binary vectors known to those skilled in the art include pTOK162 (Japanese patent Appl. (Kokai) No. 4-222527, EP 504,869, EP 604,662, and U.S. Pat. No. 5,591,616) and pTOK233 (Komari, Plant Cell Reports 9, 303 (1990); Ishida et al., Nature Biotechnology 14, 745 (1996)). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and the nucleic acid of interest to be transformed into the plant. The nucleic acid to be inserted into the plant genome is typically located between the two border sequences of the T region. Super-binary vectors of the invention can be constructed having the features described above for pTOK162. The T-region of the super-binary vectors and other vectors for use in the invention are constructed to have restriction sites for the insertion of the genes to be delivered. Alternatively, the DNA to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

In plants stably transformed by Agrobacteria-mediated transformation, the nucleotide sequence of interest is incorporated into the plant nuclear genome, typically flanked by at least one T-DNA border sequence and generally two T-DNA border sequences.

Plant cells may be transformed with Agrobacteria by any means known in the art, e.g., by co-cultivation with cultured isolated protoplasts, or transformation of intact cells or tissues. The first uses an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

Methods of introducing a nucleic acid into a plant can also comprise in vivo modification of genetic material, methods for which are known in the art. For example, in vivo modification can be used to substantially delete (“knockout”) a DEP1 gene, optionally followed by insertion of a nucleic acid encoding a dep1 polypeptide; to modify a DEP1 gene so that it expresses a dep1 polypeptide (as discussed in detail in section II above, for example, to truncate a DEP1 gene); and/or to modify a dep1 gene.

For example, one or more nucleotides may be deleted from, added to and/or replaced in vivo in a DEP1 nucleic acid encoding a native DEP1 polypeptide, resulting in a frame shift or nonsense mutation. In representative embodiments, the DEP1 gene encoding a native DEP1 polypeptide is modified in vivo such that the resultant polypeptide is truncated and functions as a dep1 polypeptide that increases NUE in a plant. For example, one or more nucleotides may be deleted from, added to and/or replaced in the DEP1 gene encoding the native DEP1 polypeptide, resulting in a nonsense mutation that gives rise to a truncated form of the native DEP1 polypeptide. Other modifications including substitutions, insertions, deletions and/or additions can further be made to the DEP1 or dep1 gene using in vivo modification techniques.

A further aspect of the invention encompasses a method of introducing a nucleic acid encoding a dep1 polypeptide into a plant, the method comprising replacing the nucleic acid (e.g., gene) that encodes the native DEP1 polypeptide with an isolated nucleic acid (e.g., an isolated nucleic acid that encodes a dep1 polypeptide).

Suitable methods for in vivo modification include the techniques described in Gao et. al., Plant J. 61, 176 (2010); Li et al., Nucleic Acids Res. 39, 359 (2011); U.S. Pat. Nos. 7,897,372 and 8,021,867; U.S. Patent Publication No. 2011/0145940 and in International Patent Publication Nos. WO 2009/114321, WO 2009/134714 and WO 2010/079430. For example, one or more transcription affector-like nucleases (TALEN) and/or one or more meganucleases may be used to modify and/or replace the nucleic acid (e.g., gene) encoding the native DEP1 polypeptide. In representative embodiments, the method comprises cleaving the nucleic acid encoding the native DEP1 polypeptide with a TALEN and/or a meganuclease and providing a nucleic acid that is homologous to at least a portion of the nucleic acid encoding the native DEP1 polypeptide, such that homologous recombination occurs and results in the deletion of one or more nucleotides from the nucleic acid, the insertion of one or more nucleotides into the nucleic acid and/or the replacement of one or more nucleotides in the nucleic acid. In other embodiments, the method comprises excising the nucleic acid encoding the native DEP1 polypeptide from the plant genome with TALENs and/or meganucleases and, optionally, providing a nucleic acid encoding an isolated dep1 polypeptide, such that homologous recombination occurs and results in the insertion of the nucleic acid encoding the dep1 polypeptide into the location previously occupied by the nucleic acid encoding the native DEP1 polypeptide.

Protoplasts, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. I, 1984, and Vol. II, 1986). Essentially all plant species can be regenerated from cultured cells or tissues, including but not limited to, all major species of sugar-cane, sugar beet, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

Alternatively, transgenic plants may be produced using the floral dip method (See, e.g., Clough and Bent (1998) Plant Journal 16:735-743, which avoids the need for plant tissue culture or regeneration. In one representative protocol, plants are grown in soil until the primary inflorescence is about 10 cm tall. The primary inflorescence is cut to induce the emergence of multiple secondary inflorescences. The inflorescences of these plants are typically dipped in a suspension of Agrobacterium containing the vector of interest, a simple sugar (e.g., sucrose) and surfactant. After the dipping process, the plants are grown to maturity and the seeds are harvested. Transgenic seeds from these treated plants can be selected by germination under selective pressure (e.g., using the chemical bialaphos). Transgenic plants containing the selectable marker survive treatment and can be transplanted to individual pots for subsequent analysis. See Bechtold, N. and Pelletier, G. Methods Mol Biol 82, 259-266 (1998); Chung, M. H. et al. Transgenic Res 9, 471-476 (2000); Clough, S. J. and Bent, A. F. Plant J 16, 735-743 (1998); Mysore, K. S. et al. Plant J 21, 9-16 (2000); Tague, B. W. Transgenic Res 10, 259-267 (2001); Wang, W. C. et al. Plant Cell Rep 22, 274-281 (2003); Ye, G. N. et al. Plant J., 19:249-257 (1999).

The particular conditions for transformation, selection and regeneration can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine what is an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

The present invention is more particularly described in the following examples that are intended as illustrative only since numerous modifications and variations therein will be apparent to those skilled in the art.

### Example 1

### Identification of Gene Associated with High Efficiency Nitrogen Utilization in Rice

It has been established that crop varieties vary in their ability to utilize available nitrogen, as determined by measuring grain yield per unit of available nitrogen in the soil (sometimes referred to as “physiological” NUE). The genetic basis of NUE in rice rests presently at the level of the identification of a number of quantitative trait loci, with no understanding of the nature of the genes presumed to underlie them.

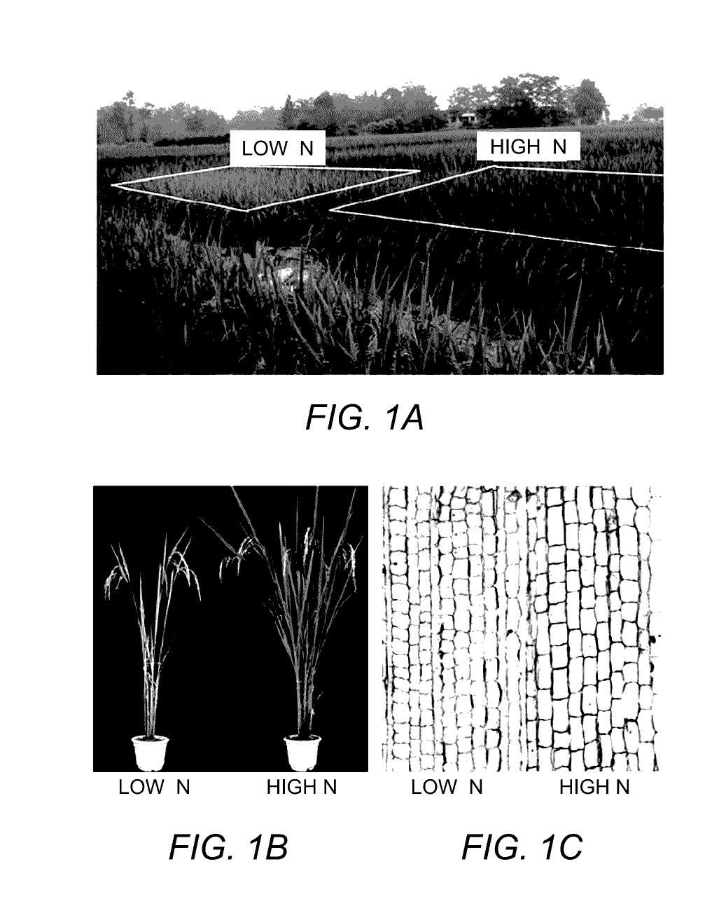

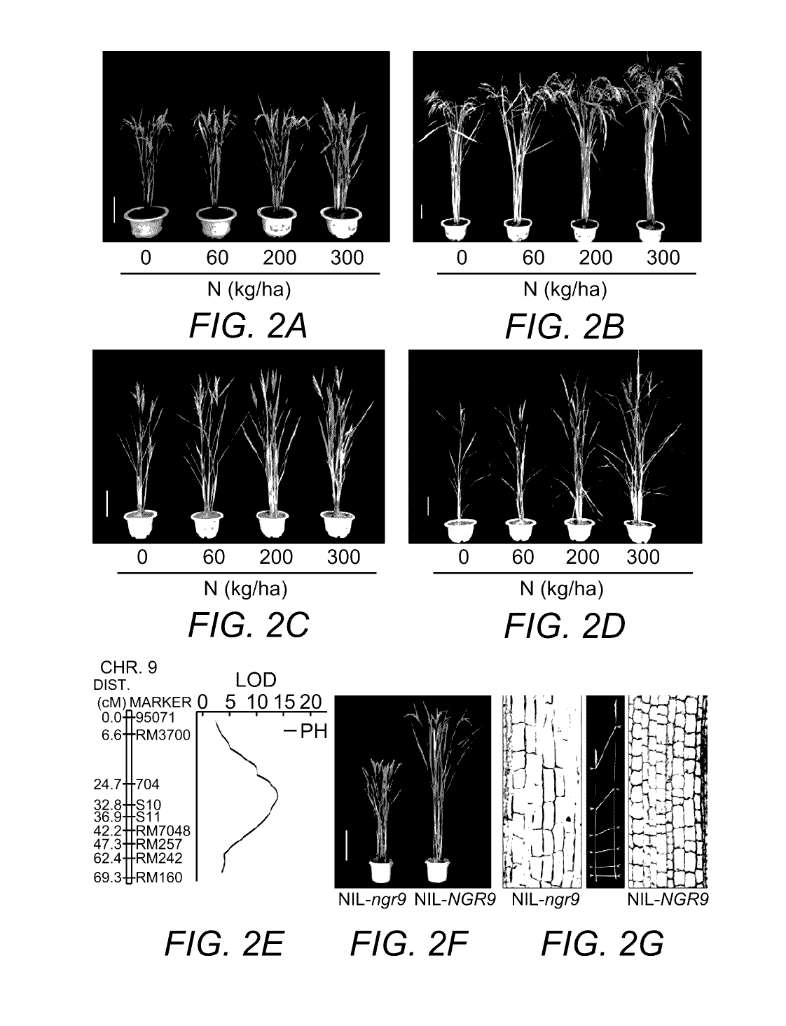

In a screen of 62 rice accessions based on the measurement of plant architecture and harvest index (“HI”; the ratio of grain yield to above-ground dry matter biomass), we have observed that while the application of nitrogenous fertilizer increased tillering ability and plant height by promoting cell proliferation and elongation (FIG. 1), the response varied from accession to accession (Table 1). Notably the japonica variety Qianzhonglang2 (QZL2) was rather insensitive (FIG. 2A; Table 2), while the indica variety Nanjing6 (NJ6) was highly sensitive (FIG. 2B); this difference was manifested by QZL2's consistently superior HI (Table 2).

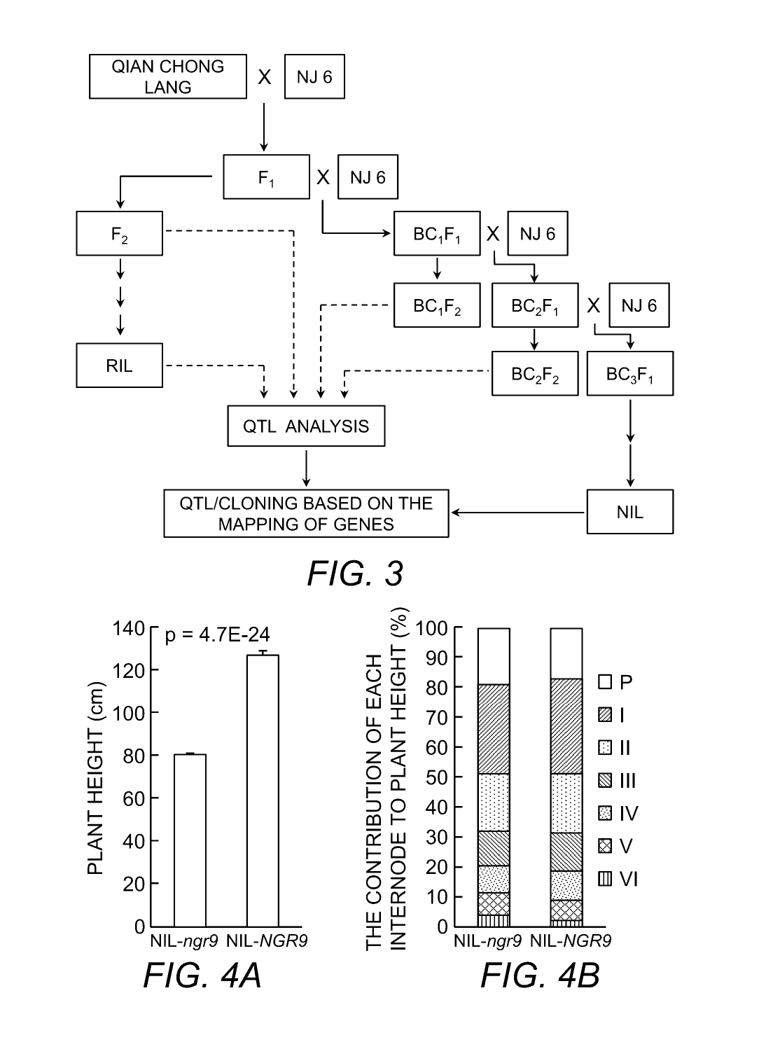

Based on the NJ6/QZL2 differential response, the HI and growth response of a set of 226 recombinant inbred lines (RILs) bred from the cross NJ6×QZL2 were measured. The NJ6×QZL2 hybrid was inbred for six generations, and then a near isogenic recombinant inbred line was constructed by back crossing for three generations (FIG. 3). One of the resulting RILs (D04) behaved in a similar manner to QZL2, while another (D22) proved to be sensitive to nitrogenous fertilizer (FIG. 2C, D; Table 2). A subsequent genetic analysis based on a BC₂F₂ population derived from the cross QZL2×D22 identified qNGR9, a major quantitative locus responsible for nitrogen growth response mapping on chromosome 9 (FIG. 2E). As a step toward mendelizing this locus, the near isogenic line (NIL) pair NIL-NGR9 and NIL-ngr9 was then developed; these lines only differ with respect to a short chromosomal segment (including qNGF9) in an otherwise homogenous background of QZL2. Comparisons based on these two NILs showed that the dominant qngr9 allele from QZL2 was associated with semidwarfism (FIG. 2F), but the NIL lines did not differ from one another with respect to contribution of each internode to the overall plant height (FIG. 4). Although the NIL-ngr9 internode cells were longer than those in NIL-NGR9, the internode length in NIL-ngr9 was less than in NIL-NGR9 plants (FIG. 2G).

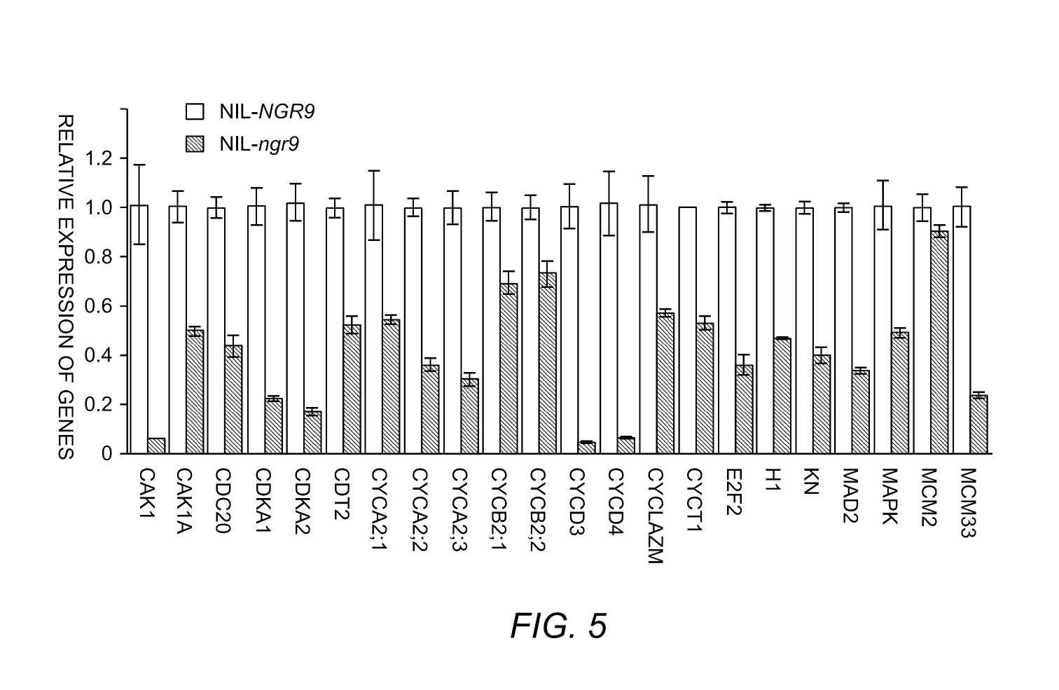

Further studies showed that the transcription of key genes determining cell cycle time, such as CDKA1, CYCD3 and E2F2, was significantly less in NIL-ngr9 than in NIL-NGR9 plants (FIG. 5). Thus, we concluded that the qngr9 allele functions as a negative regulator of cell proliferation.

### Example 2

### Characterization of the qNGR9 Allele

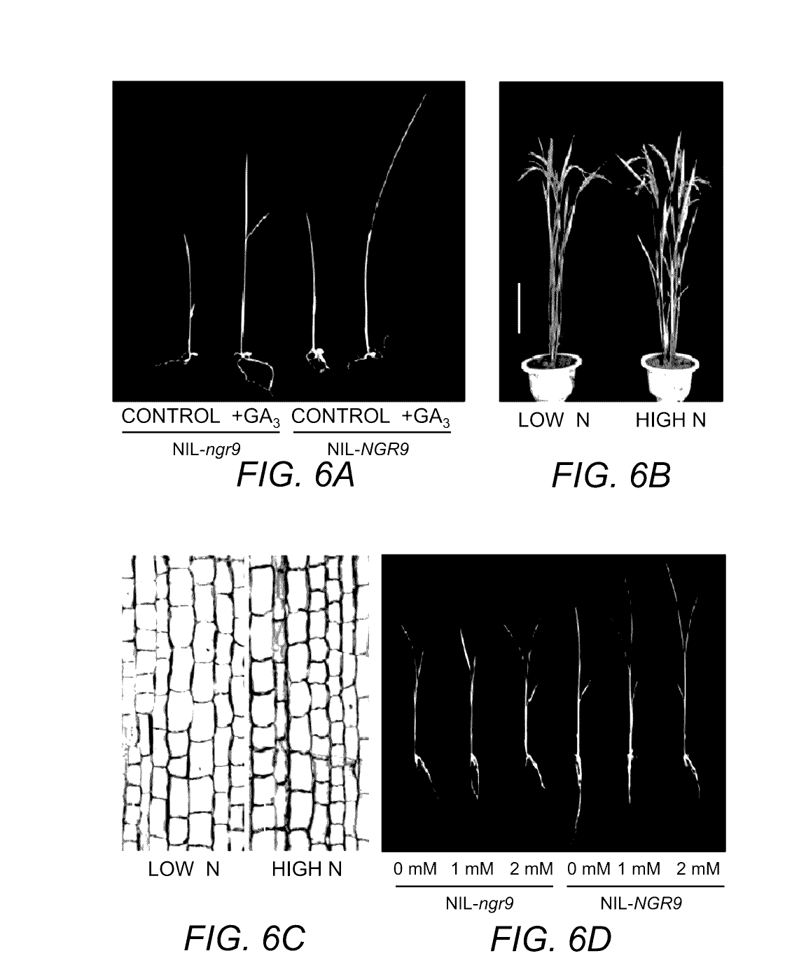

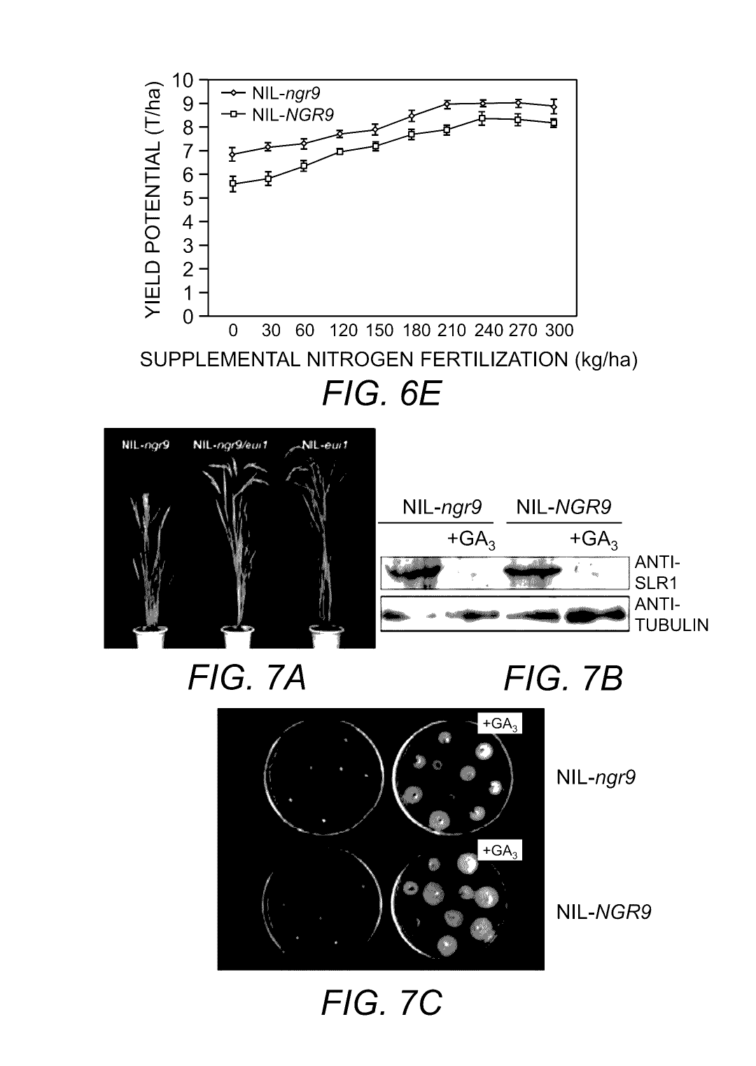

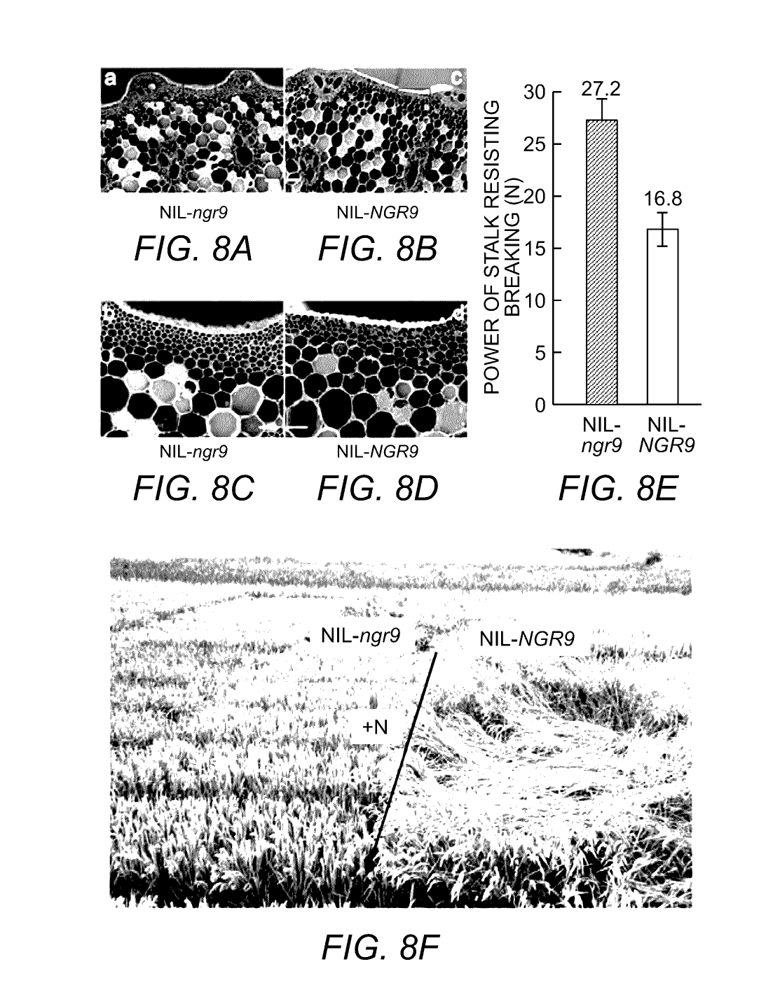

The Green Revolution semidwarf genes in both rice and wheat are involved in the synthesis and signaling of the phytohormone gibberellin (GA). NIL-ngr9 seedlings proved to be less sensitive to exogenously supplied GA than NIL-NGR9 (FIG. 6A), while the eui/ngr9 combination responded similarly to the eui mutant with respect to internode elongation due to increasing GA levels (FIG. 7). GA is known to trigger the degradation of the DELLA repressor protein and thereby to promote plant growth. However, NIL-ngr9 and NIL-NGR9 plants did not differ from one another with respect to either GA-mediated DELLA degradation or alpha-amylase production (FIG. 7). Thus it appeared that the qngr9 allele does not affect GA signaling Neither culm length nor cell number was enhanced in NIL-ngr9 plants by the application of nitrogenous fertilizer (FIG. 6B, C). The internode sclerenchyma cell walls in NIL-ngr9 were thicker than in NIL-NGR9 plants, and the bending moment at breaking, a parameter for physical strength of the culm in NIL-ngr9 was significantly enhanced (FIG. 8). While NIL-NGR9 plants lodged in response to high doses of nitrogenous fertilizer, those of NIL-ngr9 remained upright (FIG. 8). Thus, the qngr9 allele appears to enhance culm strength, resulting in improved lodging resistance.

### Example 3

### qngr9 Allele Confers Adaptation to Low Nitrogen Conditions

Plants have evolved a number of strategies to sustain their growth under conditions of limiting nitrogen availability. In a hydroponic system, the roots of NIL-NGR9 seedlings grew longer under lower nitrogen conditions, whereas shoot biomass accumulation was suppressed (FIG. 6D). In contrast, the root to shoot biomass ratio of NIL-ngr9 plants was unaffected by the reduction in nitrogen availability (FIG. 6D). Briefly, after priming the seeds of NIL-ngr9 and NIL-NGR9 at 37° C., the buds were cultured in water for 4 days, then ½ nutrient solution was added (Na₂SO₄.10H₂O, 88.022 mg/L; KH₂PO₄, 24.8 mg/L; K₂SO₄, 31.859 mg/L; MgSO₄.7H₂O, 134.82 mg/L; CaCl₂.2H₂O, 53.702 mg/L; Fe-EDTA, 7.346 mg/L; Na₂SiO₃H₂O, 465.139 mg/L; NH₄NO₃, 160 mg/L; H₂BO₃, 2.86 ug/L; CuSO₄. 5H₂O, 0.08 ug/L; ZnSO₄.7H₂O, 0.22 ug/L; MnCl₂.4H₂O, 1.81 ug/L; H₂MoO₄.H₂O, 0.09 ug/L; diluted with MES to pH 5.6), cultured for 3 days, then replaced with fresh nutrient solution and left for another 3 days. After that, the seedlings were separated into treatment groups and cultured in different nitrogen concentrations (0, 1, 2, 4, 6 mM). The nutrient solution was replaced every 7 days thereafter, and the pH was adjusted every 2 days (to pH 5.6). Nine days later, pictures were taken (FIG. 6D) and the height of the seedlings was measured (data not shown). (Under culture conditions of 15 h light/9 h dark, constant temperature 22° C., light intensity 65 μM m⁻² s⁻¹). These studies confirmed that the growth and root to shoot biomass ratio of NIL-ngr9 rice seedlings is not sensitive to nitrogen fertilizer, whereas the growth of NIL-NGR9 seedlings was.

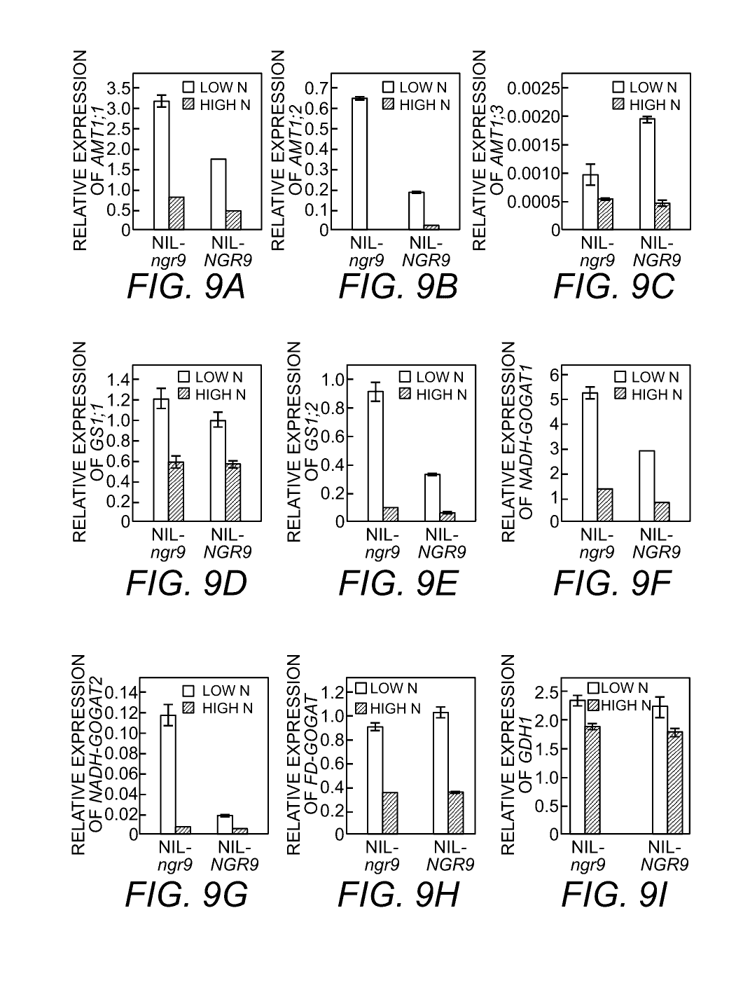

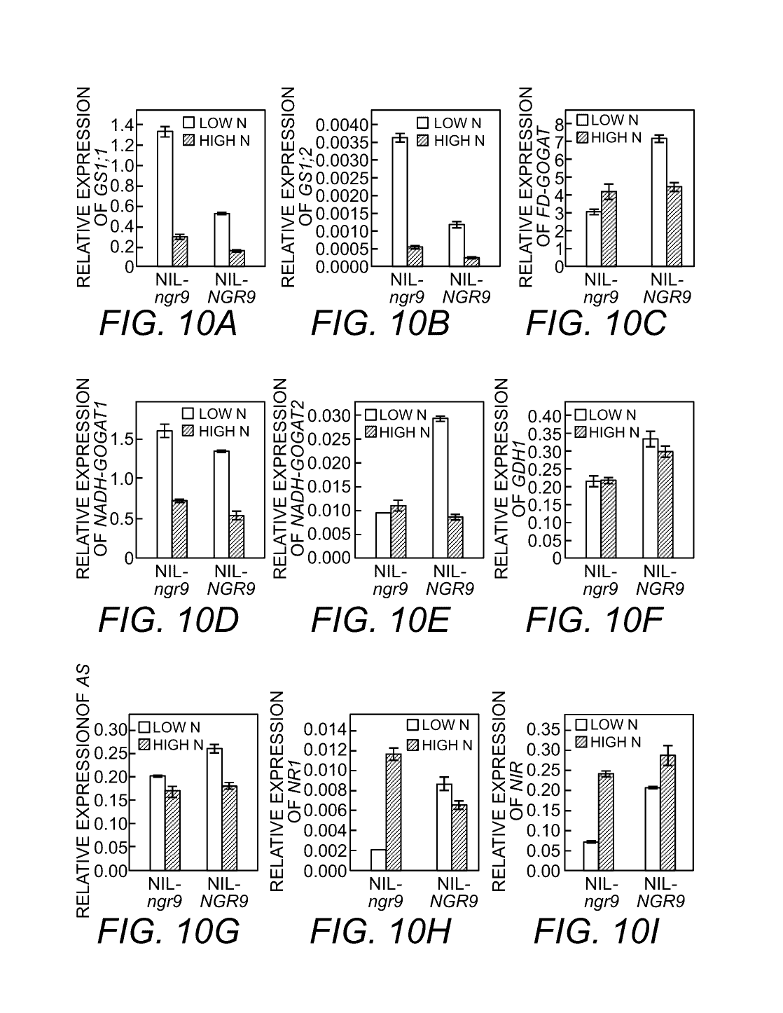

In paddy rice, the plant roots experience anaerobic conditions and, as a result, are forced to utilize ammonium rather than nitrate as a source of inorganic nitrogen. Ammonium is taken up by the rice root via high affinity transporters, and is subsequently assimilated into glutamine (Gln) by the coupled reaction of glutamine synthetase (GS) and glutamine synthase (GOGAT). Cytosolic GS1;2 and plastidic NADH-GOGAT1 are largely responsible for the primary assimilation of ammonium. When the expression of genes involved in the uptake and assimilation of ammonium in the roots was explored via qRT-PCR, it was clear that the expression level of genes encoding the ammonium transporters (AMT1;1 and 1;2), glutamine synthetase (GS1;2) and the two NADH-dependent glutamate synthases (NADH-GOGAT1 and 2) were upregulated in a nitrogen deficient environment in both NIL-ngr9 and NIL-NGR9, but the transcript abundance of each gene was markedly higher in NIL-ngr9 plants (FIG. 9). During the vegetative phase of plant growth, nitrogen is accumulated by the plant, and during its reproductive phase, most of it (in rice, ˜80%) is remobilized and translocated to the developing seed. Cytosolic GS1;1 and NADH-GOGAT1 are the major enzymes responsible for this process. We observed that GS1;1 was upregulated in NIL-ngr9 plants grown under both high and low nitrogen conditions (FIG. 10), consistent with the observed positive correlation between grain yield and GS1 activity in maize. These observations are in agreement with the nonresponsiveness of tillering shown by NIL-ngr9 plants grown under low levels of available nitrogen (Table 2), and suggest that the qngr9 allele contributes to adaptation to low soil nitrogen conditions.

### Example 4

### qngr9 Allele Results in Improved Yield Performance and Increased Agricultural NUE

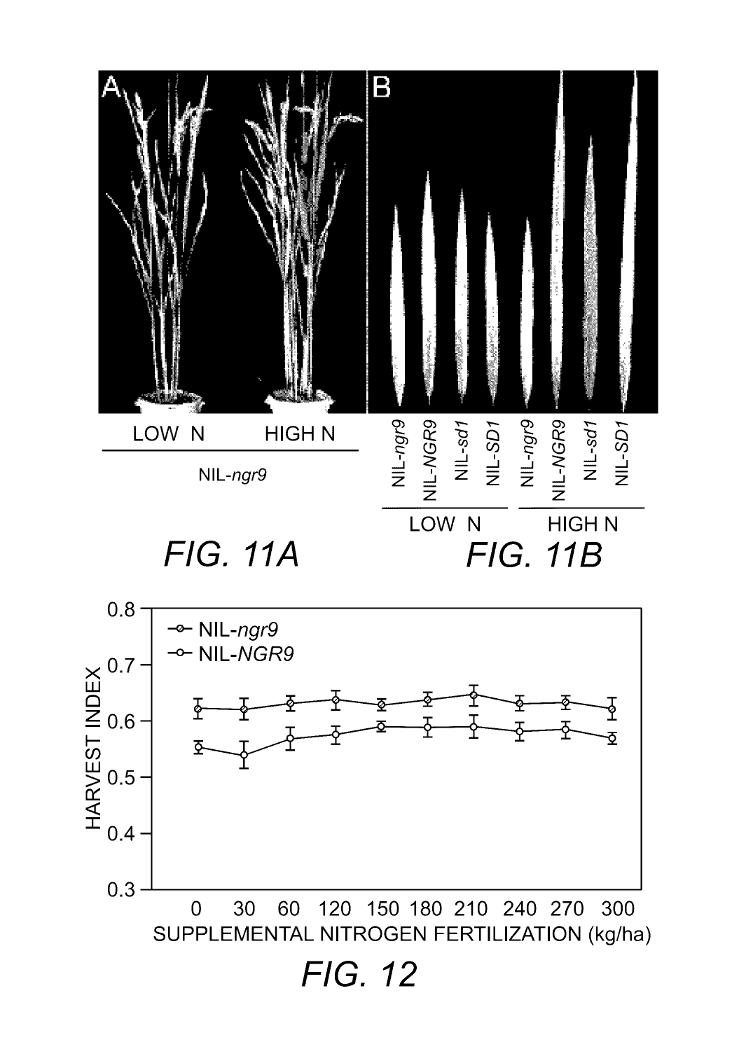

When NIL-ngr9 rice is grown with the application of different levels of nitrogen, plant height does not vary significantly (FIG. 11A). The effect of different applications of nitrogen on the growth of rice leaves is shown in FIG. 11B. Higher nitrogen concentrations can improve leaf growth of NIL-NGR9 rice, but the impact of nitrogen fertilizer concentration on leaf growth of NIL-ngr9 rice is not significant.

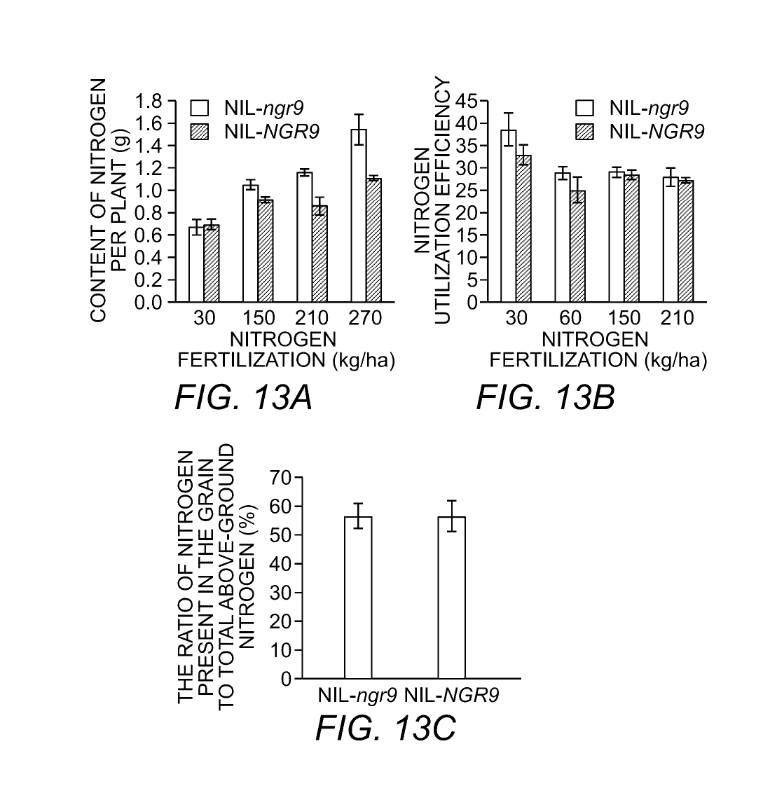

The performance of field-grown NIL-ngr9 and NIL-NGR9 plants was compared with respect to grain yield and HI. Although the NILs came to heading simultaneously, NIL-ngr9 out-performed for both HI and grain yield (FIG. 6E; FIG. 12). The above-ground nitrogen content per plant was higher in NIL-ngr9 than in NIL-NGR9 plants, but there was no discernible difference in the ratio of grain dry mass to above-ground nitrogen (a measure of physiological nitrogen utilization efficiency) or the proportion of nitrogen present in the grain to total above-ground nitrogen (FIG. 13). However, when standard commercial cultivation practices (150 kg/ha nitrogen) were applied, the grain yield recorded by NIL-ngr9 was ˜9% higher than that of NIL-NGR9 (FIG. 6E). To achieve this same yield, NIL-NGR9 required the application of an additional 60 kg/ha nitrogen (40% more), and at this level, NIL-ngr9 still out-yielded NIL-NGR9 by ˜14% (FIG. 6E). Thus, the presence of the qngr9 allele results in increased NUE.

### Example 5 qngr9 Corresponds to the dep1 Allele

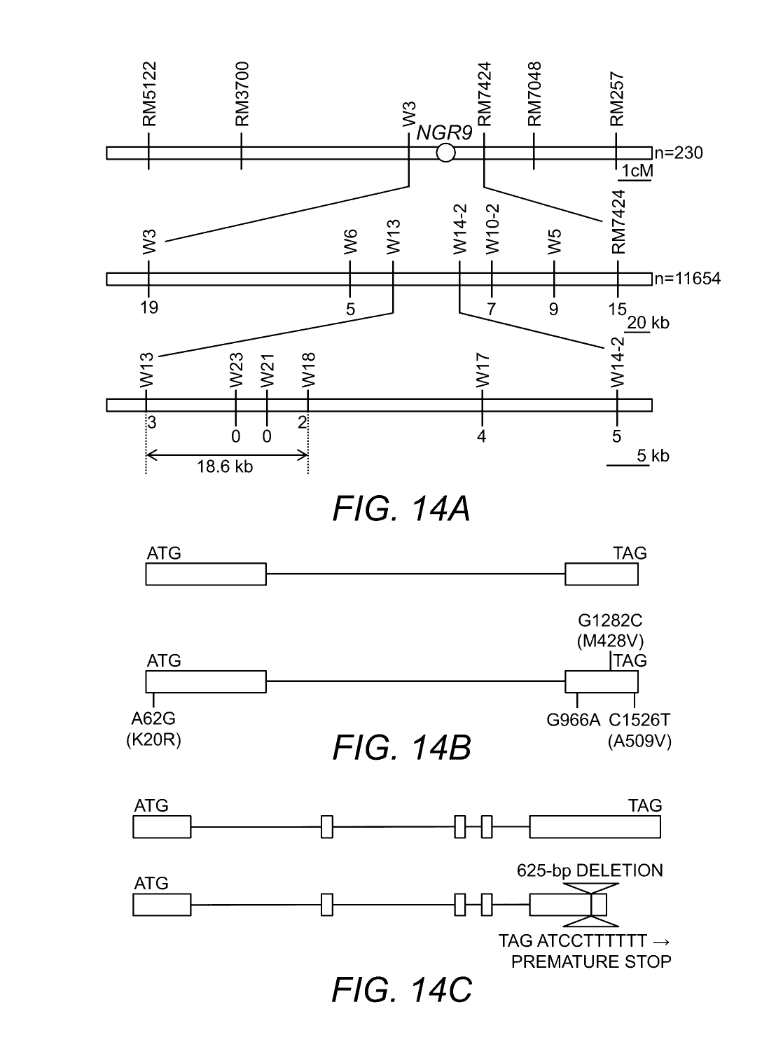

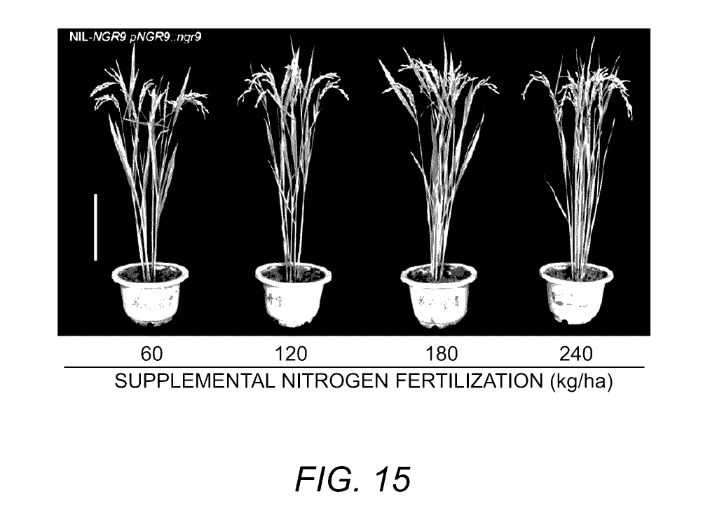

Fine mapping was based on the genotypic analysis of 11,654 BC₃F₂ segregants, which allowed the candidate region to be narrowed to a ˜18.6 kbp segment flanked by the markers W13 and W18 (FIG. 14A). Sequence comparisons of this region present in the two mapping parents and cv. Nipponbare indicated the existence of two polymorphic predicted open reading frames (Os09g0441700 and Os09g0441900). The former encodes a putative cytochrome P450 protein, and the mapping parent sequences differ from one another by one synonymous (G996A) and three replacement (A62G, G1282C and C1526T) polymorphisms (FIG. 14). The polymorphisms within Os09g0441900 between NIL-ngr9 and NIL-NGR9 match those already defined for the variants at DENSE ERECT PANICLE 1 (“DEP1”; FIG. 14). To confirm that DEP1 is synonymous with qNGR9, a genomic fragment containing either the Os09g0441700 or the Os09g0441900 sequence cloned from NIL-ngr9 was transformed into NIL-NGR9 via Agrobacterium-mediated transformation. The dep1 transgenic plants were semi-dwarfed in stature, and they did not respond to the addition of nitrogenous fertilizer (FIG. 15). However, the transgenic plants expressing the NIL-ngr9 Os09g0441700 allele did not differ in phenotype from the non-transgenic NIL-NGR9 plants; the supply of nitrogenous fertilizer promoted an increase in stem and leaf elongation (data not shown). These results indicate that dep1 is synonymous with qngr9. Transgenic NIL-NGR9 plants expressing qngr9/dep1 had semi-dwarf stature and their growth was insensitive to nitrogenous fertilizer application (FIG. 15). These results indicate that the qngr9/dep1 allele has two contrasting effects on plant architecture: First, it represses cell division during vegetative growth and, secondly, it enhances cell proliferation during the reproductive stage.

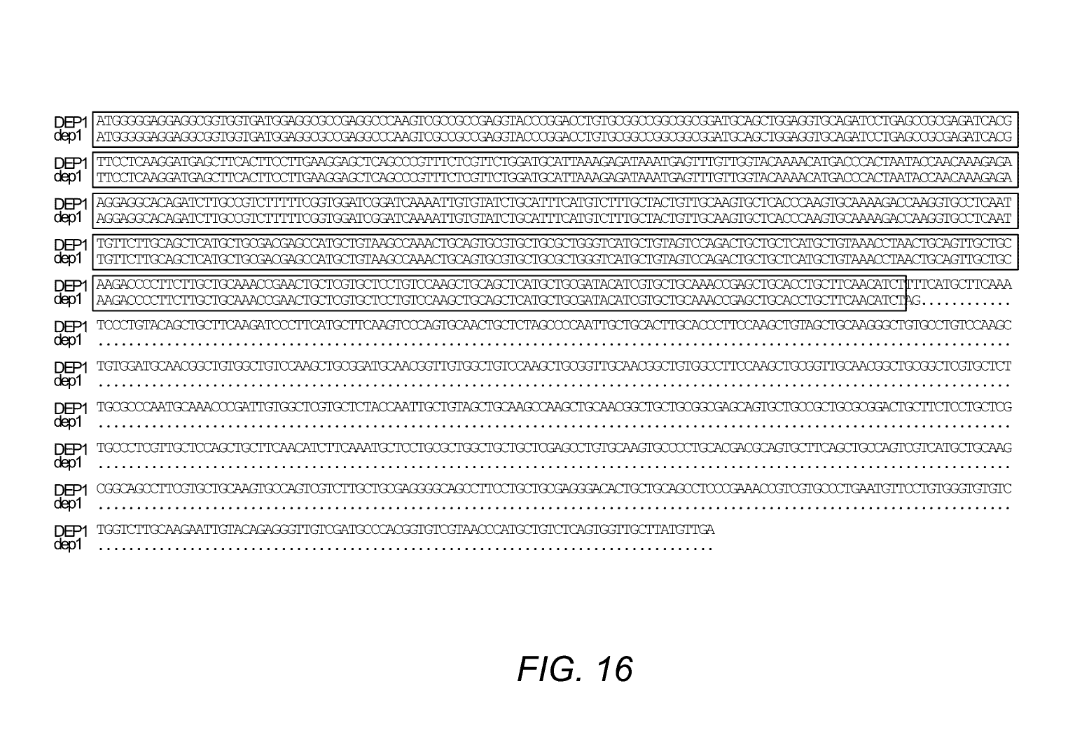

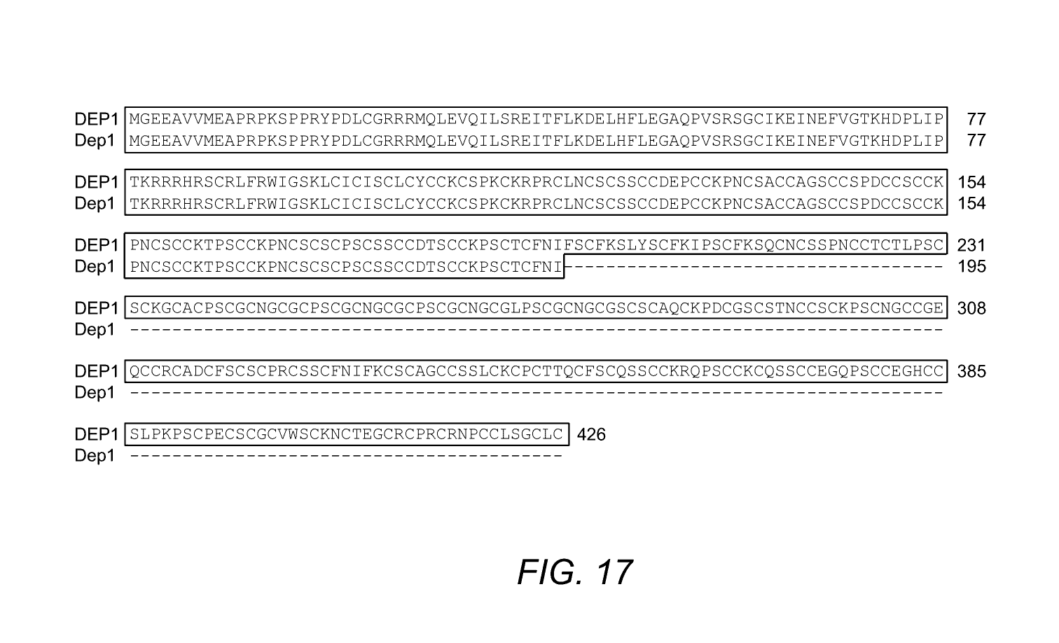

DEP1 encodes a protein that includes a TNFR/NGFR cysteine-rich domain. Sequence analysis indicates that the nucleotide sequence of the high nitrogen efficiency gene ngr9 is the same as the dense and erect panicle gene dep1. The results of sequence comparison showed that the DEP1 cDNA contains 1281 bp, while dep1 has 588 bp at the 5′ end and lacks 696 bp at the 3′ end (FIG. 16); the protein sequence comparison of dep1/DEP1 showed that the DEP1 protein has 426 amino acids, while dep1 has only 195 amino acids at the N end and lacks 231 amino acids at the C end (FIG. 17).

A schematic of the DEP1 protein, indicating the premature stop codon in dep1 is shown in FIG. 18. The nucleotide (SEQ ID NO: 8) and amino acid (SEQ ID NO: 19) sequences of DEP1 are shown in FIGS. 16 and 17, along with the nucleotide (SEQ ID NO: 1) and amino acid (SEQ ID NO: 9) sequences of dep1.

### Example 6

### Cellular Localization of DEP1

The subcellular localization of DEP1 was evaluated following Agrobacterium-mediated transient expression of a DEP1-GFP fusion protein in tobacco cells.

Briefly, the method is as follows:
1) Transfer activated Agrobacterium tumefaciens EHA 105 (purchased from the Biovector Science Lab, China) into 50 ml liquid LB (containing 50 ug/ml kanamycin and 25 ug/ml rifampicin), and shake the bacterial suspension at 220 rpm for one night at 28° C.
2) Centrifuge a bacterial suspension at 5000 g to precipitate bacterial pellets at room temperature, and resuspend bacterial pellets with 10 mM MgCl₂, 10 mM MES-KOH, pH 5.7, 150-200 μM injection buffer solution Acetosyringone (purchased from Sigma);
3) Dilute the bacterial suspension with injection buffer solution to achieve OD600 of 0.5, 1, and 1.5 respectively;
4) Leave the bacterial suspensions at room temperature for 2-4 hours;
5) Mix the above-mentioned bacterial suspensions, and inject the mixed bacterial suspension into the lower surface of the tobacco blade with a 1-2 ml syringe; two to five days later, remove the blade and observe it under a fluorescent microscope.

Observations under the fluorescent microscope indicate that the NGR9-GFP fusion protein has both cytomembrane localization and cell nucleus localization (FIG. 19).

### Example 7

### Greater Photosynthetic Efficiency of NIL-ngr9 Plants

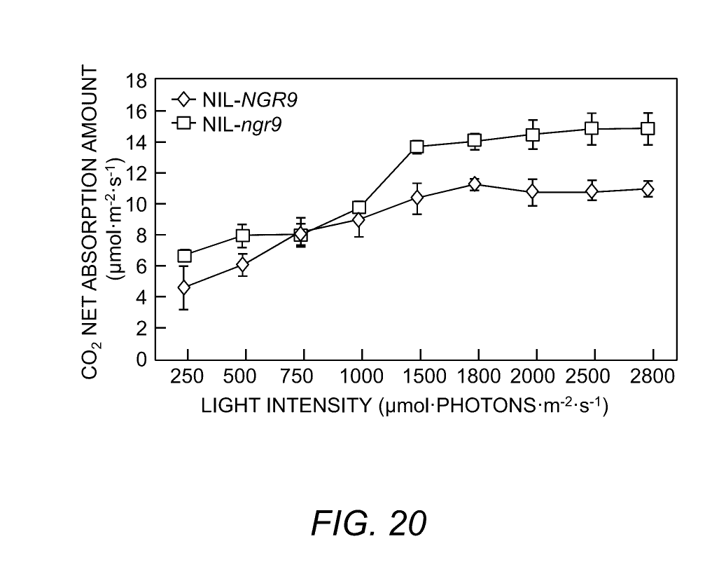

Seventy days after transplanting, rice plants are in the secondary branch phase. Photosynthetic efficiency of NIL-ngr9 and NIL-NGR9 plants was measured from 10:00 AM to 12:00 Noon. The middle parts of the top two leaves from two different plants were picked for each measurement. Using a photosynthetic apparatus L1-6400 (LI-CORINC., Lincoln, Nebr., USA) set at different light intensities (250, 500, 750, 1000, 1500, 1800, 2000, 2500, 2800 μmol photons m⁻² sec⁻¹), the net absorption (μmol m⁻² sec⁻¹) of CO₂ was measured. The results indicate that NIL-ngr9 shows greater photosynthetic efficiency than NIL-NGR9 (FIG. 20).

### Example 8

### Identification of Orthologs in Other Plant Species

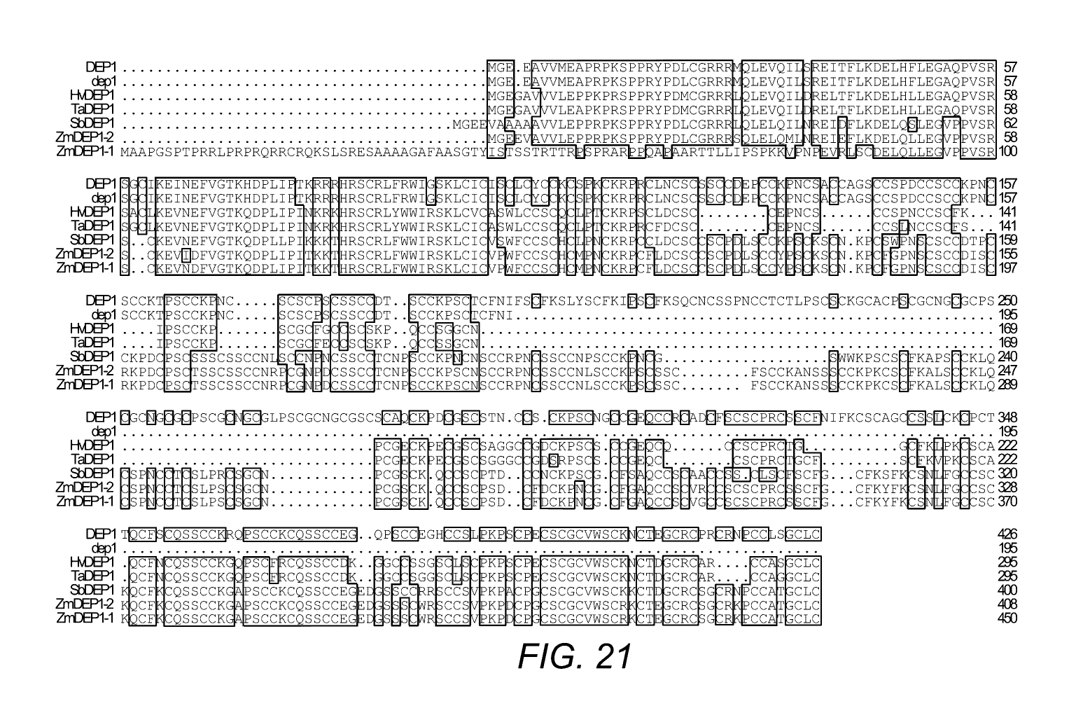

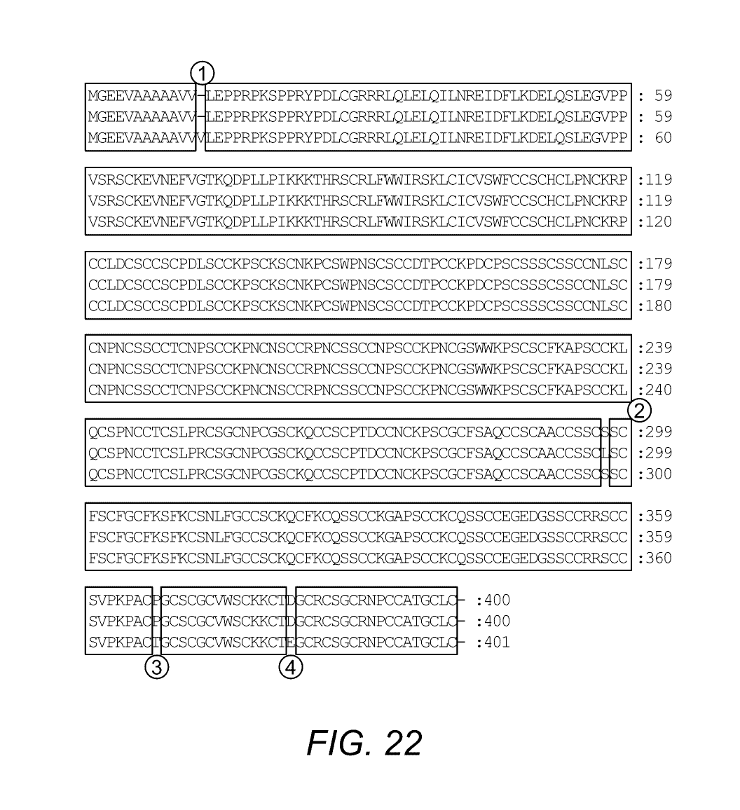

Using the sequence of rice dep1 as a probe, and the Basic Logical Alignment Search Tool (BLAST) in the NCBI database, orthologous cDNA sequences from the bread wheat variety Triticum aestivum (TaDep1; SEQ ID NO: 2), barley (HvDep1; SEQ ID NO: 4), maize (ZmDep1-1 and ZmDep1-2; SEQ ID NOS: 5 and 6) and sorghum (SbDep1; SEQ ID NO: 7) have been identified. An alignment of the corresponding proteins is shown in FIG. 21. The percent amino acid similarities between the encoded proteins is as follows: the similarity between TaDEP1 (SEQ ID NO: 10) and rice DEP1 (SEQ ID NO: 19) is 49.42%, the similarity between TaDEP1 (SEQ ID NO: 19) and rice dep1 (SEQ ID NO: 9) is 44.41%; the similarity between HvDEP1 (SEQ ID NO: 12) and rice DEP1 (SEQ ID NO: 19) is 50%, the similarity between HvDEP1 (SEQ ID NO: 12) and rice dep1 (SEQ ID NO: 9) is 43.73%; the similarity between SbDEP1 (SEQ ID NO: 16) and rice DEP1 (SEQ ID NO: 19) is 51.99%, the similarity between SbDEP1 (SEQ ID NO: 16) and rice dep1 (SEQ ID NO: 9) is 33.83%; the similarity between ZmDEP1-1 (SEQ ID NO: 14) and rice DEP1 (SEQ ID NO: 19) is 43.34%, the similarity between ZmDEP1-1 (SEQ ID NO: 14) and rice dep1 (SEQ ID NO: 9) is 22.35%; the similarity between ZmDEP1-2 (SEQ ID NO: 15) and rice DEP1 (SEQ ID NO: 19) is 36.07%, the similarity between ZmDEP1-2 (SEQ ID NO: 15) and rice dep1 (SEQ ID NO: 9) is 31.13%. In addition, multiple naturally occurring amino acid variations exist in the sorghum SbDEP1 protein. There are variations at four amino acid positions in three different varieties of sorghum (SEQ ID NOS: 16-18; FIG. 22). The nucleotide (SEQ ID NO: 3) and amino acid (SEQ ID NO: 11) sequences of the dep1 ortholog from the bread wheat diploid wild progenitor Triticum urartu (TuDEP1) have previously been reported (U.S. Patent Publication No. 2011/0197305 A1).

By means of homology-based cloning, the orthologous genes TaDEP1 and HvDEP1 have been isolated from wheat and barley, respectively. Vector constructs expressing TaDEP1 and HvDEP1 from an actin promoter were used to transform Nipponbare. Plants transformed with either TaDEP1 or HvDEP1 showed a similar phenotype to rice dep1 transformed plants: semi-dwarf plants with a compact panicle and increased grain number (U.S. Patent Publication No. 2011/0197305 A1).

In addition, it has been demonstrated that the TaDEP1 gene regulates and controls panicle type in wheat. An RNAi construct was used to knockdown TaDEP1 expression in wheat; the downregulation of TaDEP1 resulted in an increase in the length of the ear, a less compact ear and a somewhat reduced number of spikelets (Huang et al., 2009 Nature Genetics 41:494-497; U.S. Patent Publication No. 2011/0197305 A1).

To further evaluate the effect of dep1 in other species, we constructed apUbi:dep1 vector and used it to produce genetically modified corn plants overexpressing the rice dep1 gene. The genetically modified corn exhibited a semi-dwarf plant type and has dark green leaves (data not shown). Using the photosynthetic apparatus L1-6400 (LI-COR Inc., Lincoln, Nebr., USA) to measure the net absorption (μ mol m⁻² sec⁻¹) of CO₂ under 1500μ mol photons m⁻² sec⁻¹ light intensity, it was observed that the photosynthetic efficiency of dep1 genetically modified corn is increased by 25.6% as compared with the control group. It was also observed that the leaf angle of the corn plants expressing dep1 was smaller. Thus, the characteristics conferred by dep1, such as a smaller leaf angle, semi-dwarf plant type, and high photosynthetic efficiency may be advantageous in improving planting density of corn and increase photosynthetic efficiency, thereby improving yield.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

TABLE 1
Rice germplasm accessions surveyed for variation in NUE

| Accession | Origin | Class | sd1 allele |
|---|---|---|---|
| Guichao2 | Guangdong, China | indica | yes |
| Aizizhan | Guangdong, China | indica | yes |
| Yeqingzhan3 | Guangdong, China | indica | yes |
| Guangluai4 | Guangdong, China | indica | yes |
| 9311 | Jiangsu, China | indica | yes |
| Minghui63 | Fujiang, China | indica | yes |
| Zhefu802 | Zhejiang, China | indica | yes |
| Erjiuqing | Zhejiang, China | indica | yes |
| Zhenshan97B | Jiangxi, China | indica | yes |
| TN-1 (Taichung Native-1) | Taiwan, China | indica | yes |
| IR8 | IRRI, Philippine | indica | yes |
| IR24 | IRRI, Philippine | indica | yes |
| IR36 | IRRI, Philippine | indica | yes |
| IR64 | IRRI, Philippine | indica | yes |
| IR65600-27 | IRRI, Philippine | indica | yes |
| IR66764-60 | IRRI, Philippine | indica | yes |
| RD23 | Thailand | indica | yes |
| Bg90-2 | Sri Lanka | indica | yes |
| Amol3 | Iran | indica | yes |
| Khazar | Iran | indica | yes |
| Nantehao | Fujiang, China | indica | no |
| Lucaihao | Fujiang, China | indica | no |
| Nanjing6 | Jiangsu, China | indica | no |
| Dabaigu | Guangxi, China | indica | no |
| Xiaohongdao | Anhui, China | indica | no |
| Aus116 | Bangladesh | indica | no |
| Aus143 | India | indica | no |
| Kasalath | India | indica | no |
| 30416 | Brazil | indica | no |
| 9177 | India | indica | no |
| 8231 | Vietnam | indica | no |
| 9148 | Thailand | indica | no |
| Laoliaqing | Jiangsu, China | japonica | no |
| Wuyujing5 | Jiangsu, China | japonica | no |
| Wuyunjing7 | Jiangsu, China | japonica | no |
| Taihuoqing | Zhejiang, China | japonica | no |
| Xiushui04 | Zhejiang, China | japonica | no |
| Xiushui11 | Zhejiang, China | japonica | yes |
| Zhongzao01 | Zhejiang, China | japonica | no |
| Zhonghua11 | Tianjing, China | japonica | no |
| Xinxiannu1 | Henan, China | japonica | no |
| Zhongxin5 | Hebei, China | japonica | no |
| Qianzhonglang2 | Liaoning, China | japonica | no |
| Weiguo | Liaoning, China | japonica | no |
| Liaohe5 | Liaoning, China | japonica | no |
| Longjing1 | Heilongjiang, China | japonica | no |
| Daohuaxiang2 | Heilongjiang, China | japonica | no |
| Jijing62 | Jilin, China | japonica | no |
| Jijing88 | Jilin, China | japonica | no |
| Yunjing36 | Yunnan, China | japonica | no |
| Dianjinyou1 | Yunnan, China | japonica | no |
| Baisenugu | Guangxi, China | japonica | no |
| Tainong54 | Taiwan, China | japonica | no |
| Tainong67 | Taiwan, China | japonica | no |
| Taizhong65 | Taiwan, China | japonica | no |
| Katy | USA | japonica | no |
| Lemont | USA | japonica | no |
| Nongken58 | Japan | japonica | no |
| Shanxin22 | Japan | japonica | no |
| Nipponbare | Japan | japonica | no |
| 19282 | Egypt | japonica | no |
| Balila | Itality | japonica | no |

TABLE 2
Effect of nitrogenous fertilizer on harvest index and plant growth

| | Nitrogen | | | |
|---|---|---|---|---|
| | fertilization | | Tiller | |
| Variety | (Kg/ha) | Plant height | numbers | Harvest index |
| QZL2 | 0 | 76.5 ± 0.6 | 8.2 ± 0.2 | 0.55 ± 0.03 |
| | 60 | 77.8 ± 04 | 8.7 ± 0.3 | 0.59 ± 0.02 |
| | 200 | 78.2 ± 0.2 | 8.9 ± 0.4 | 0.63 ± 0.01 |
| | 300 | 80.6 ± 0.4 | 9.2 ± 0.3 | 0.62 ± 0.02 |
| NJ6 | 0 | 106.3 ± 0.7 | 7.0 ± 0.3 | 0.47 ± 0.02 |
| | 60 | 111.7 ± 1.2 | 9.5 ± 0.2 | 0.46 ± 0.03 |
| | 200 | 128.3 ± 0.7 | 12.3 ± 0.4 | 0.44 ± 0.01 |
| | 300 | 136.4 ± 0.9 | 14.8 ± 0.6 | 0.45 ± 0.04 |
| RIL-D04 | 0 | 75.8 ± 1.0 | 5.6 ± 0.1 | 0.57 ± 0.03 |
| | 60 | 78.0 ± 0.7 | 5.8 ± 0.2 | 0.58 ± 0.01 |
| | 200 | 81.8 ± 0.5 | 6.0 ± 0.2 | 0.60 ± 0.02 |
| | 300 | 80.9 ± 1.2 | 6.2 ± 0.2 | 0.59 ± 0.03 |
| RIL-D22 | 0 | 82.8 ± 0.8 | 3.2 ± 0.4 | 0.48 ± 0.02 |
| | 60 | 86.3 ± 1.1 | 5.6 ± 0.3 | 0.49 ± 0.03 |
| | 200 | 102.8 ± 1.3 | 8.0 ± 0.3 | 0.48 ± 0.01 |
| | 300 | 118.1 ± 0.6 | 10.4 ± 0.5 | 0.49 ± 0.04 |

* Rice plants were grown with a distance of 20 × 20 cm in paddies under normal cultivation conditions. Data given as mean ± SE (n = 60).

1. A method of increasing nitrogen utilization efficiency (NUE) in a transgenic plant, the method comprising introducing an isolated nucleic acid encoding a dep1 polypeptide into a plant to produce a transgenic plant that expresses the isolated nucleic acid to produce the dep1 polypeptide, thereby resulting in an increased NUE in the transgenic plant as compared with a control plant.

2. The method of claim 1, wherein the method further comprises growing the plant under low nitrogen conditions.

3. The method of claim 2, wherein the method results in an increased yield of the transgenic plant under low nitrogen conditions as compared with a control plant.

4. The method of claim 2, wherein the low nitrogen conditions comprise the application of a reduced level of nitrogen fertilizer to the transgenic plant.

5. The method of claim 4, wherein the low nitrogen conditions comprise the application of 120 kilograms per hectare or less of nitrogen fertilizer.

6. The method of claim 2, wherein the low nitrogen conditions comprise growing the transgenic plant in a low nitrogen medium.

7. The method of claim 1, wherein the plant comprises in its genome the isolated nucleic acid encoding a dep1 polypeptide.

8. The method of claim 1, wherein the method comprises:
(a) introducing the isolated nucleic acid into a plant cell to produce a transgenic plant cell; and
(b) regenerating a transgenic plant from the transgenic plant cell of (a), wherein the transgenic plant comprises in its genome the isolated nucleic acid encoding a dep1 polypeptide and has increased NUE.

9. The method of claim 1, wherein the method comprises:
(a) introducing the isolated nucleic acid into a plant cell to produce a transgenic plant cell;
(b) regenerating a transgenic plant from the transgenic plant cell of (a), wherein the transgenic plant comprises in its genome the isolated nucleic acid encoding a dep1 polypeptide; and
(c) selecting from a plurality of the transgenic plants of (b) transgenic plant having increased NUE.

10. The method of claim 7, wherein the method further comprises obtaining a progeny plant derived from the transgenic plant, wherein the progeny plant comprises in its genome the isolated nucleic acid encoding a dep1 polypeptide and has increased NUE.

11. The method of claim 1, wherein the introducing is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, or a combination thereof.

12. The method of claim 1, wherein the isolated nucleic acid comprises an expression cassette comprising a nucleotide sequence encoding the dep1 polypeptide operably associated with a promoter operable in a plant cell.

13. The method of claim 1, wherein the isolated nucleic acid comprises:
(a) a nucleotide sequence that encodes the amino acid sequence of any one of SEQ NOS: 9-13; or
(b) a nucleotide sequence that encodes an amino acid sequence that is at least 70% similar to the amino acid sequence of any one of SEQ ID NOS: 9-13 and provides increased NUE to a transgenic plant expressing the same.

14. The method of claim 1, wherein the isolated nucleic acid comprises a nucleotide sequence that encodes the amino acid sequence of any one of SEQ ID NOS: 9-13.

15. The method of claim 1, wherein the isolated nucleic acid comprises a nucleotide sequence that encodes the amino acid sequence of SEQ ID NO: 9.

16. The method of claim 1, wherein the isolated nucleic acid comprises a nucleotide sequence encoding the dep1 polypeptide, the nucleotide sequence selected from the group consisting of:
(a) a nucleotide sequence of any one of SEQ NOS: 1-4;
(b) a nucleotide sequence that is at least 70% identical to a nucleotide sequence of any one of SEQ ID NOS: 1-4 and provides increased NUE to a transgenic plant expressing the same;
(c) a nucleotide sequence that hybridizes to the complete complement of the nucleotide sequence of any one of SEQ ID NOs: 1-4 under stringent conditions comprising a wash stringency of 50% Formamide with 5×Denhardt's solution, 0.5% SDS and 1×SSPE at 42° C. and provides increased NUE to a transgenic plant expressing the same; or
(d) a nucleotide sequence that differs from the nucleotide sequence of any of (a) to (c) due to the degeneracy of the genetic code.

17. The method of claim 1, wherein the nucleotide sequence is the nucleotide sequence of any one of SEQ ID NOS: 1-4.

18. The method of claim 1, wherein the nucleotide sequence is the nucleotide sequence of SEQ ID NO:1.

19. The method of claim 1, wherein the plant is a monocotyledonous plant.

20. The method of claim 1, wherein the plant is rice, maize, wheat, barley, sorghum, oat, rye, or sugar cane.
